(19) United States
(12) Patent Application Publication
(10) Pub. No.: US 2025/0049305 A1
(43) Pub. Date: Feb. 13, 2025

(54) Steerable instrument for endoscopic or invasive applications

(71) Applicant: Fortimedix Assets II B.V., Geleen (NL)

(72) Inventor: Mattheus Hendrik Louis THISSEN, Swalmen (NL)

(73) Assignee: Fortimedix Assets II B.V., Geleen (NL)

(21) Appl. No.: 18/719,298

(22) PCT Filed: Dec. 13, 2022
(86) PCT No.: PCT/NL2022/050718

§ 371 Date: Jun. 13, 2024

(30) Foreign Application Priority Data
Dec. 14, 2021 (NL) 2030128

(51) Int. Cl. A61B 1/005; A61B 17/00

(52) U.S. Cl. CPC A61B 1/0051; A61B 2017/00305

(57) ABSTRACT

A steerable instrument for endoscopic and/or invasive, e.g. gastroscopic or colonoscopic, applications, such as in surgery, having:
a cylindrical element with a proximal end part, a distal end part including a flexible distal portion, an intermediate part between the proximal and distal end parts, and at least one longitudinal element for transferring forces from the proximal end part to the flexible distal portion to control bending of the latter;
a tangential spacer arranged adjacent the longitudinal element;
a second cylindrical element coaxial with the cylindrical element, with a proximal end part, a distal end part including a flexible distal portion, and an intermediate part there between;
a radial spacer attached to the second cylindrical element and protruding into an aperture in the tangential spacer;
wherein movement of the tangential spacer both in a longitudinal direction and in a tangential direction of the instrument is confined by the radial spacer.

### FIELD OF THE INVENTION

The present invention relates to a steerable instrument for endoscopic and/or invasive type of applications, such as in surgery. The instrument according to the invention is in particular suited for colonoscopic and/or gastroscopic applications. The steerable instrument according to the invention can be used in both medical and non-medical applications. Examples of the latter include inspection and/or repair of mechanical and/or electronic hardware at locations that are difficult to reach. Hence, terms used in the following description such as endoscopic application or invasive instrument, must be interpreted in a broad manner.

### BACKGROUND ART

Transformation of surgical interventions that require large incisions for exposing a target area into minimal invasive surgical interventions, i.e. requiring only natural orifices or small incisions for establishing access to the target area, is a well-known and ongoing process. In performing minimal invasive surgical interventions, an operator such as a physician, requires an access device that is arranged for introducing and guiding invasive instruments into the human or animal body via an access port of that body. In order to reduce scar tissue formation and pain to a human or animal patient, the access port is preferably provided by a single small incision in the skin and underlying tissue. In that respect the possibility to use a natural orifice of the body would even be better. Furthermore, the access device preferably enables the operator to control one or more degrees of freedom that the invasive instruments offer. In this way, the operator can perform required actions at the target area in the human or animal body in an ergonomic and accurate manner with a reduced risk of clashing of the instruments used.

Surgical invasive instruments and endoscopes through which these instruments are guided towards the target area are well-known in the art. Both the invasive instruments and endoscopes can comprise a steerable instrument that enhances its navigation and steering capabilities. Such a steerable instrument preferably comprises a proximal end part including at least one flexible zone, a distal end part including at least one flexible zone, and a rigid intermediate part, wherein the steerable instrument further comprises a steering arrangement that is adapted for translating a deflection of at least a part of the proximal end part relative to the rigid intermediate part into a related deflection of at least a part of the distal end part.

Furthermore, the steerable instrument preferably comprises a number of co-axially arranged cylindrical elements including an outer element, an inner element and one or more intermediate elements depending on the number of flexible zones in the proximal and distal end parts of the instrument and the desired implementation of the steering members of the steering arrangement, i.e. all steering members can be arranged in a single intermediate cylindrical element or the steering members are divided in different sets and each set of steering members is arranged, at least in part, in a different or the same intermediate member. In most prior art devices, the steering arrangement comprises conventional steering cables with, for instance, sub 1 mm diameters as steering members, wherein the steering cables are arranged between related flexible zones at the proximal and distal end parts of the instrument. Other steering units at the proximal end, like ball shaped steering units or robot driven steering units, may be applied instead.

However, as steering cables have many well-known disadvantages, it is preferred to avoid them and to implement the steering members by one or more sets of longitudinal elements that form integral parts of the one or more intermediate cylindrical elements. Each of the intermediate cylindrical elements including the longitudinal elements can be fabricated either by using a suitable material addition technique, such as injection molding or plating, or by starting from a cylindrical element and then using a suitable material removal technique, such as laser cutting, photochemical etching, deep pressing, conventional chipping techniques such as drilling or milling or high-pressure water jet cutting systems. Of the aforementioned material removal techniques, laser cutting is very advantageous as it allows a very accurate and clean removal of material under reasonable economic conditions. Further details regarding the design and fabrication of the abovementioned steerable instrument and the steering arrangement thereof have been described for example in WO 2009/112060 A1, WO 2009/127236 A1, U.S. Ser. No. 13/160,949, and U.S. Ser. No. 13/548,935 of the applicant, all of which are hereby incorporated by reference in their entirety.

Steerable invasive instruments typically comprise a handle that is arranged at the proximal end part of the steerable instrument for steering the instrument and/or for manipulating a tool that is arranged at the distal end part of the steerable instrument. Such a tool can for example be a camera, a manual manipulator, e.g. a pair of scissors, forceps, or manipulators using an energy source, e.g. an electrical, ultrasonic or optical energy source.

Assembling a steerable instrument having an intermediate cylindrical element in a steerable instrument is quite difficult since the longitudinal elements provide the intermediate cylindrical element with a very much decreased bending stiffness. The intermediate cylindrical element with the longitudinal steering elements may deform in a very uncontrolled manner. It easily loses its geometrical coherence. Manipulating the intermediate cylindrical element during and after providing the longitudinal elements therein can become problematic, especially while assembling the steerable instrument. This is quite cumbersome when carrying out the assembly, and it may also cause damage to the intermediate cylindrical element. Such damage generally yields a steerable instrument with deteriorated performance, which is very much undesired.

These issues are addressed by WO 2016/089202 A1, disclosing a steerable instrument and a method of manufacturing such. The method provides an efficient and well-controlled method of manufacturing a steerable instrument. In order to avoid displacement, deformation and/or disordering of the longitudinal elements, elements are provided for keeping adjacent longitudinal elements and/or in a set relationship with respect to one another during assembly of the instrument. These elements may be flexible elements, which have a permanent attachment to the respective longitudinal element(s). Alternatively, the elements may be releasably or temporarily attached to the longitudinal elements, for example by fracture points or by elements referred to as fracture elements. These temporary or releasable attachments can be caused to fracture, break, or otherwise release after assembly of the instrument. An embodiment hereof is reproduced in FIGS. 8a and 8b of the present application.

While the use of the fracture elements as described by WO 2016/089202 A1 offers many advantages during assembly of the instrument, after assembly they remain within the open space between neighboring longitudinal elements. Thereby, relative movement of the two neighboring longitudinal elements is limited. Furthermore, if not breaking properly at each fraction point, the relative movement of the longitudinal elements may be even more obstructed, e.g. limited to half of its intended extension. Still further, if the operator of the instrument applies a force to the instrument causing it to bend to such an extent that a relative movement of the neighboring longitudinal elements exceeds the range set by the length of the open space, the fracture element may be forced into an undesired position, such as into longitudinal slits separating adjacent longitudinal elements, or into a space below or above the longitudinal elements, where it might get stuck or otherwise causing severe damage to the instrument.

Another problem which might occur during use of the steerable instruments relates to inadvertent clamping of the longitudinal elements between the inner and outer layers. In these steerable instruments, the longitudinal elements need to be flexible in at least those portions of the instrument that should allow bending relative to the longitudinal axis of the instrument, both at the proximal and distal ends. These longitudinal elements are often located between an adjacent outer and adjacent inner cylindrical element. When bending these flexible zones of the instrument, in each such zone these longitudinal elements bend together with bendable portions of the outer and inner cylindrical element. However, sometimes the bending of such zone causes the bending outer and bending inner cylindrical element to clamp the longitudinal element between the outer and inner cylindrical elements such that it is difficult to move the longitudinal elements any further in the longitudinal direction. This effect may also be caused/increased by different longitudinal elements arranged on top of each other in the bendable portions.

WO 2019/009710 A1 describes a steerable instrument having a construction aimed at preventing clamping of the longitudinal element between the outer and inner cylindrical elements. Several different embodiments are presented for creating a distance between different cylindrical elements, such that clamping of longitudinal elements there between is prevented. In some embodiments, as reproduced in FIGS. 9a-9c of the present application, radial spacers are realized by small lip shaped portions formed in one cylindrical element which, after coaxial alignment of the cylindrical element with other cylindrical elements, are bent in a radial direction such as to touch another cylindrical element, thereby creating a distance there between.

However, the presence of such radial spacers formed by bent lip shaped portions may put restrictions on the degree to which the instrument can be bent. As shown in FIGS. 9a-9a, the width of the longitudinal elements vary along their length. The edges of these longitudinal element portions limit the open space between adjacent longitudinal elements, thereby limiting relative movement of adjacent longitudinal elements if edges of broadened portions thereof come into contact with a radial or tangential spacer.

Furthermore, if in WO 2019/009710 A1 the different cylindrical elements are not aligned with sufficient accuracy, and/or if the direction and/or point of application of the force applied for bending the lip shaped portion is not accurate, the lip shaped portion may not be bent as intended. The bending of the lip shaped portion may inadvertently result in the lip shaped portion not touching the inner cylinder, whereby the desired radial spacing may not be achieved, and/or in the lip shaped portion abutting one or both of the adjacent longitudinal elements, causing friction during use of the instrument.

Some locations to be examined and/or operated in a body need specifically designed instruments. In particular if these locations are located at a relatively large distance from a suitable point of entry into the body, the instrument needs sufficient length, and a high degree of flexibility may be required in the intermediate part of the instrument, while, obviously, maintaining functionality of the instrument.

### SUMMARY OF THE INVENTION

It is an object of the invention to provide a steerable instrument for endoscopic and/or invasive type of applications where at least one of the above mentioned problems are solved or at least reduced.

In particular, it is an object of the invention to provide a steerable instrument having improved bending properties, e.g. a steerable instrument which can handle higher bending and/or torsional stress or forces.

This is achieved by a steerable instrument as claimed in claim 1.

In another embodiment, the object of the invention is to provide a steerable instrument which can be more easily assembled and/or manufactured, in particular wherein the instrument is less prone to be associated with faults, errors or imperfections caused by the manufacturing process.

This is achieved by a cylindrical element as claimed in claim 17.

Embodiments of the invention are claimed in dependent claims.

In a first aspect a steerable instrument for endoscopic and/or invasive type of applications, such as in surgery, is provided, the instrument comprising:
a first cylindrical element comprising a first proximal end part, a first distal end part including at least one first flexible distal portion, and a first intermediate part between said first proximal end part and said first distal end part, and at least one longitudinal element arranged for transferring a force from said first proximal end part to said at least one first flexible distal portion and thereby control bending of said at least one first flexible distal portion;
at least one tangential spacer arranged tangentially adjacent to said at least one longitudinal element in an open space between said at least one longitudinal element and an other portion of the first cylindrical element;
a second cylindrical element arranged coaxially with said first cylindrical element, and comprising a second proximal end part, a second distal end part including at least one second flexible distal portion, and a second intermediate part between said second proximal end part and said second distal end part, corresponding to and substantially aligned with said first proximal end part, said first distal end part and said first intermediate part of said first cylindrical element, respectively;
at least one radial spacer attached to the second cylindrical element and protruding into an aperture in the tangential spacer;
such that movement of the tangential spacer in both a longitudinal direction and in a tangential direction of the steerable instrument is confined by said at least one radial spacer; and, optionally
at least one further radial spacer attached to the second cylindrical element and protruding in the open space at a longitudinal end of the at least one tangential spacer.

The first cylindrical element may also be referred to as an intermediate cylindrical element or layer. The instrument may comprise more than one such intermediate cylindrical layer, depending on, e.g., the number of flexible distal zones in, and/or construction requirements of, the steerable instrument.

In order to avoid confusion and to clearly distinguish between corresponding features of the various cylindrical elements, such as proximal and distal end parts, flexible distal portions, etc., these are referred to with reference to the cylindrical element in which they are provided. For example, the feature “first proximal end part” refers to the proximal end part of the first cylindrical element. This feature may alternatively be referred to as “proximal end part of the first cylindrical element”.

The at least one longitudinal element is separated from tangentially adjacent or near-by located parts of the first cylindrical element by the at least one tangential spacer. In some embodiments, more than one longitudinal element, e.g. a plurality of longitudinal elements are provided, as well as a plurality of tangential spacers. In such embodiments, adjacent longitudinal elements are separated from one another by at least one tangential spacer located therebetween.

That is, the expression “an other portion of the first cylindrical element”, used herein above, may encompass any parts of the cylindrical element extending in the longitudinal direction, i.e. adjacent the at least one longitudinal element, but not forming a longitudinal element arranged for transferring force, as well as any further longitudinal element(s) in an embodiment comprising a plurality of longitudinal elements.

The at least one longitudinal element provides a controlled bending of the at least one first flexible distal zone of the instrument. If a plurality of longitudinal elements are provided, these may be arranged to control bending of a plurality of distal zones. At the proximal end part of the steerable instrument, an operator, such as a surgeon, or a computer or a robot, controls bending of the one or more flexible distal zones, by applying a force to the at least one longitudinal element or at least some of the plurality of longitudinal elements. The at least one longitudinal element, extending between the proximal and distal end parts of the instrument, transfers this force to the distal end part, whereby one or more of the flexible distal zones are bent. When more than one flexible distal zones are provided, these are individually controlled, such that bending of the distal end part of the instrument can be achieved in accordance with the intention of the operator.

To this end, at the proximal end part, the longitudinal elements may be connected to linear actuators controlled by e.g. a computer or control unit like a robot. Alternatively, the proximal end part of the first cylindrical element may include at least one flexible proximal portion, arranged such that the bending of this flexible proximal portion results in a controlled bending of a corresponding flexible distal portion. Such a flexible portion may be implemented by corresponding hinges in the cylindrical element in the form of a suitable slit shaped pattern, or by a ball shaped steering unit or a tiltable plate, as a person skilled in the art will understand.

The second cylindrical element may also be referred to as an outer cylindrical element or layer. The second cylindrical element may also be an intermediate layer within the assembled instrument, whereby a further outer layer is provided.

The different cylindrical elements or layers are coaxially aligned, such that corresponding flexible portions are aligned along the cylindrical axis. As will be described below, a third cylindrical element may be provided as an inner cylindrical element or a further intermediate cylindrical element.

The intermediate part, extending between the proximal end part and the distal end part, is flexible throughout its extension, such as to be able to bend in different directions. This bending is in general not directly controllable by the operator, but takes place in correspondence with e.g. the geometry of the space into which the instrument is introduced, e.g. as the instrument, during insertion into and/or passage through a space, such as the colon or intestine, touches walls or other obstructions thereof. The flexibility of the intermediate region must be such that no damage is caused to the internal walls of the passage through which it passes through. A substantially fully bendable and/or flexible instrument is provided, suitable for various applications where the distal end portion of the steerable instrument has to reach a position which is difficult to reach and/or located at some distance from a point of entry into a body, such as a human body. Examples of such applications include colonoscopic and gastroscopic applications.

The tangential spacer is arranged to provide and/or maintain a distance between the at least one longitudinal element and other parts of the first cylindrical element, and/or between adjacent longitudinal elements in embodiments comprising a plurality of longitudinal elements, and to limit movement of the at least one longitudinal element in tangential, circumferential and/or transversal direction.

The tangential spacer is provided with at least one aperture, into which the radial spacer protrudes, thereby confined in its movement with respect to the at least one longitudinal element in the direction parallel and antiparallel to the extension of the longitudinal element and in a tangential direction of the first cylindrical element.

The tangential spacer may be formed by an element which prior to assembly of the instrument is attached to the at least one longitudinal element via one or more fracture locations or fracture elements, which are fractured after assembly of the instrument, e.g. in a manner as described in WO 2016/089202 A1.

In embodiments wherein a plurality of longitudinal elements are provided, these are arranged with a gap or distance between adjacent longitudinal element in the longitudinal direction, forming an open space. In each of these open spaces, one or more of said tangential spacers can be arranged. The longitudinal elements preferably have a shape and/or dimension such that movement of longitudinal elements relative to one another and relative to the tangential spacers is not limited by the longitudinal elements themselves or portions thereof.

By the at least one radial spacer, movement of the tangential spacer with respect to the second cylindrical element is limited and/or confined. The tangential spacer can be said to be maintained in place by the radial spacer. Hence, the distance between neighboring longitudinal elements is maintained by the tangential spacer, while relative movement of adjacent longitudinal elements along one another is enabled, and while unintended displacement of the spacer, e.g. in the form of the tangential spacer sliding on top of or underneath the first cylindrical element, is prevented.

The radial spacer protrudes into, and preferably extends through, the open space between the at least one longitudinal element and other parts of the cylindrical element adjacent thereto, or between adjacent longitudinal elements. It is attached to, and/or forms part of, e.g. by being formed by a part of, the second cylindrical element. Preferably, it is flexible and/or has at least some degree of elasticity or flexibility with respect to the second cylindrical element. The radial spacer may be a radial spacer of the second aspect of the invention.

Preferably, a plurality of tangential spacers and a plurality of radial spacers are provided between adjacent longitudinal elements.

The tangential spacer is provided with an aperture and the radial spacer protrudes at least partly into said aperture. Thereby, one radial spacer is sufficient for limiting a movement of the tangential spacer both in the longitudinal and tangential direction of the steerable instrument.

The first cylindrical element may be a first cylindrical tube, and both the at least one longitudinal element and the at least one tangential spacer may be portions of the first cylindrical tube.

The second cylindrical element may be a second cylindrical tube. The at least one radial spacer and the at least one further radial spacer may be formed by a lip shaped portion of the second cylindrical tube and protruding from the second cylindrical tube. The lip shaped portion may have a lip shaped portion width and the aperture an aperture width, wherein the lip shaped portion width is either smaller than the aperture width or is configured such that it clamps the tangential spacer when bent into the aperture.

The at least one longitudinal element has a length in the longitudinal direction and a width in a direction substantially perpendicular to said longitudinal direction in said first intermediate part, wherein preferably said width is substantially constant throughout said length. This further reduces the risk of the tangential spacer sliding on top of or underneath the longitudinal element. If the at least one longitudinal element has a uniform width throughout at least the extension of the intermediate part of the first cylindrical element, such that the distance between adjacent longitudinal elements is substantially constant in this part, the movement of adjacent longitudinal elements with respect to one another will not be limited by interactions between the longitudinal elements and the tangential or radial spacers.

The instrument may further comprise a third cylindrical element arranged coaxially with said first cylindrical element and said second cylindrical element, wherein said first cylindrical element is arranged between said second cylindrical element and said third cylindrical element, and wherein at least one of the following applies: said at least one radial spacer or said at least one further radial spacer is arranged to abut said third cylindrical element, is engaged with said third cylindrical element, and is attached to said third cylindrical element. This arrangement contributes to the mechanical stability of the instrument.

The at least one radial spacer provides a distance between the second and third cylindrical elements, which is preferably larger than the thickness of the longitudinal elements and the tangential spacer in the radial direction. Clamping of the at least one longitudinal element between the second and third cylindrical elements during bending of the flexible zone is thereby prevented.

The third cylindrical element may advantageously be a third cylindrical tube provided with a at least one recess or aperture, and wherein said at least one radial spacer or said at least one further radial spacer protrudes into said at least one recess or aperture. By this arrangement, the second and third cylindrical elements are fixed, or locked, with respect to one another in axial, radial and tangential direction. The radial spacer or said at least one further radial spacer may comprise a first part having a first width and a second part having a second width which is smaller than said first width, and wherein said second part protrudes into said recess or aperture. Thereby, the relative fixation of the second and third cylindrical elements may be improved.

In some embodiments, the third cylindrical tube may be provided with a plurality of recesses or apertures, wherein each of the at least one radial spacer and the at least one further radial spacer protrude into a respective one of the plurality of recesses or apertures. In such embodiment, both the at least one radial spacer and the at least one further radial spacer may comprise the first and the second parts.

The third cylindrical element comprises a third proximal end part, a second distal end part including at least one third flexible distal portion, and a third intermediate part between said third proximal end part and said third distal end part, corresponding to and substantially aligned with, respectively, said first proximal end part, said first distal end part and said first intermediate part of said first cylindrical element.

In general, the third cylindrical element forms an inner cylindrical element, said first cylindrical element forms an intermediate cylindrical element, and said second cylindrical element forms an outer cylindrical element. This provides for easy manufacturing when the radial spacer is provided by a lip shaped portion in the second cylindrical element, which lip shaped portion is bent in a radial direction. Alternatively, the second cylindrical element may form an inner cylindrical element and the third cylindrical element an outer cylindrical element.

The at least one radial spacer may advantageously be formed by a lip shaped portion protruding from said second cylindrical element. This lip shaped portion may be formed in the second cylindrical element e.g. by laser cutting, and, after assembly and alignment of the cylindrical elements, be bent in the radial direction such as to form the radial spacer. When the second cylindrical element forms an outer cylindrical element, this bending is performed from the outside, in a direction directed radially inwards.

The lip shaped portion may be formed in a second wall of said second cylindrical element such as to be attached thereto via a beam having a beam axis extending substantially perpendicular to said longitudinal direction;

wherein said lip shaped portion is defined by a first slot in said second wall, said first slot extending around said lip shaped portion except for where the lip shaped portion is attached to said beam;
said first slot further extending in a direction substantially parallel to said beam axis on either side of said lip shaped portion at one side of said beam;
wherein said beam is defined by a second slot provided in said second wall in a direction substantially parallel to said beam axis and at another side of said beam.

The steerable instrument may be substantially flexible throughout its length. In that case, said cylindrical element, said second cylindrical element, and, where present, said third cylindrical element are provided with one or more flexible portions throughout their lengths.

The steerable instrument may comprise a plurality of tangential spacers and a plurality of radial spacers as well as a plurality of longitudinal elements, wherein between each pair of adjacent longitudinal elements at least one tangential spacer and at least one radial spacer are provided. That is, tangential spacers and associated radial spacers are provided in each open gap between adjacent longitudinal elements in the intermediate part of the first cylindrical element. This provides a steerable instrument having high flexibility.

Alternatively, the steerable instrument comprises a plurality of tangential spacers, a plurality of radial spacers, and a plurality of longitudinal elements,
wherein said plurality of longitudinal elements comprises a first longitudinal element, a second longitudinal element, and a third longitudinal element,
wherein, in said first intermediate part,
a first open space is formed between said first longitudinal element and said second longitudinal element,
a second open space is formed between said second longitudinal element and said third longitudinal element,
wherein in the first open space at least one tangential spacer and at least one radial spacer are provided; and
wherein in the second open space an extended tangential spacer is provided, which extended tangential spacer extends substantially throughout the second open space.
   Hence, in this embodiment, the combination of tangential spacers and radial spacers are not provided in each open space formed between adjacent longitudinal elements, but only in every other open space. In the remaining open spaces a second tangential spacer is provided, which second tangential spacer extends substantially throughout the open space. This provides a steerable instrument having higher stability than if the combination of tangential and radial spacers are provided in each open space formed between adjacent longitudinal elements.

According to a second aspect of the invention a cylindrical element for use in a steerable instrument is provided, said cylindrical element extending along a cylindrical axis and comprising a wall, wherein at least one lip shaped portion is formed in said wall,
wherein said lip shaped portion is attached to said wall via a beam having a beam axis, said beam axis arranged at an angle to said cylindrical axis;
wherein said lip shaped portion is defined by a first slot in said wall, said first slot extending around said lip shaped portion except for where the lip shaped portion is attached to said beam;
said first slot further extending in a direction substantially parallel to said beam axis on either side of said lip shaped portion at one side of said beam; and
wherein said beam is further defined by a second slot formed in said wall in a direction substantially parallel to said beam axis and at another side of said beam. Said angle is larger than 0°, preferably larger than 45° and smaller or equal to 90°, and more preferably 90°.

Thereby, the bending direction of the lip shaped portion is defined with high accuracy, as the risk of the lip shaped portion having a direction deviating from the radial direction decreases. If the force for bending the lip shaped portion is applied perpendicular to the wall of the cylindrical element, the lip shaped portion will bend around the beam axis. Abutment and/or friction between the radial spacers and the adjacent longitudinal elements during use of the instrument can be avoided, as the radial spacer has a well-defined position and/or orientation.

The cylindrical element according to the second aspect may be used as the second cylindrical element in the steerable instrument of the first aspect, whereby the lip shaped portion forms the radial spacer.

The lip shaped portion may comprise a first part having a first width in a direction substantially parallel to said beam axis and a second part having a second width in a direction substantially parallel to said beam axis, wherein said first part is arranged between said beam and said second part, and wherein said second width is smaller than said first width. The second part is advantageously used to engage with and/or be inserted into an aperture or recess of a further, e.g. third, cylindrical element arranged coaxial with the cylindrical element provided with the lip shaped portion. Thereby, a stable fixation of the cylindrical elements may be provided.

In general, the lip shaped portion is symmetrical with respect to said beam axis and an axis substantially perpendicular to said beam axis.

The beam axis may extend in a direction substantially perpendicular to said cylindrical axis. This is preferred when the cylindrical element is used for providing radial spacers in a steerable instrument of the first aspect when the longitudinal elements extend in a direction parallel to the cylindrical axis of the first cylindrical element.

Alternatively, the beam axis may extend in a direction oriented at a different angle to the cylindrical axis of the cylindrical element. This is advantageous when the cylindrical element is used for providing radial spacers in a steerable instrument of the first aspect in an embodiment where the longitudinal elements extend in a spiraling or helical arrangement with respect to a cylinder axis of the first cylindrical element. In this case, the beam axis is preferably oriented substantially perpendicular to the longitudinal direction at the location of the radial spacer.

In general, a plurality of lip shaped portions are provided in the cylindrical element.

The lip shaped portion of the second aspect is advantageously used as the lip shaped portion, or radial spacer, of the first aspect.

In a third aspect, a method of manufacturing a cylindrical element for use in a steerable instrument, said cylindrical element extending in a direction of a cylinder axis, said method comprising:
providing a piece of material for forming a wall of said cylindrical element;
forming a first slot in said material such that at least one lip shaped portion is formed in said material and attached thereto via a beam having a beam axis, wherein said first slot extends around said lip shaped portion, except for where the lip shaped portion is attached to said beam, and further extending in a direction substantially parallel to said beam axis on either side of said lip shaped portion and at one side of said beam;
forming a second slot in said wall, said second slot extending in a direction substantially parallel to said beam axis and at another side of said beam;
   wherein after said manufacturing said one or more lip shaped portions are located in said wall of said cylindrical element.

This cylindrical element is preferably the cylindrical element of the second aspect.

The beam axis may extend in a direction at an angle to said cylinder axis. Said angle is larger than 0°, preferably larger than 45° and smaller or equal to 90°, and more preferably 90°.

The piece of material may be provided in the form of a cylindrical element, or may be rolled into a cylindrical element after forming said first and second slots.

The beam axis may extend in a direction substantially perpendicular to said cylindrical axis. Alternatively, the beam axis may extend in a different direction. Effects and applications hereof have been described above with respect to the cylindrical element of the second aspect.

In a fourth aspect a method of assembling a steerable instrument is provided, the method comprising:
providing a first cylindrical element comprising a first proximal end part, a first distal end part including at least one first flexible distal portion, and a first intermediate part between said first proximal end part and said first distal end part;
forming at least one longitudinal element extending between said first proximal end part and said first distal end part and arranged for transferring a force from said first proximal end part to said at least one first flexible distal portion and thereby control bending of the at least one flexible distal portion;
forming at least one tangential spacer being attached to said at least one longitudinal element by at least one releasable attachment;
providing a second cylindrical element comprising a second proximal end part, a second distal end part including at least one second flexible distal portion, and a second intermediate part between said second proximal end part and said second distal end part;
forming at least one lip shaped portion defined by a first slot in a second wall of said second cylindrical element in said second intermediate region;
providing a third cylindrical element comprising a third proximal end part, a third distal end part including at least one third flexible distal portion, and a third intermediate part between said third proximal end part and said third distal end part;
sliding said cylindrical elements into one another, such that said cylindrical elements are substantially coaxially aligned;
applying a force onto said at least one lip shaped portion in a direction substantially perpendicular to said second wall of said second cylindrical element, thereby bending said lip shaped portion in a radial direction;
   such that after said bending, said at least one lip shaped portion forms at least one radial spacer protruding into in an aperture in the tangential spacer;
   and such that, after said releasable attachments have been released:
   movement of said tangential spacer along both said longitudinal direction and in a tangential direction of the steerable instrument is confined by said at least one radial spacer.

The steerable instrument of the first aspect may be assembled by the method according to the fourth aspect. The various features of the method may be associated with technical effects and advantages analogous to the effects and advantages described above.

Said third cylindrical element may be provided with at least one aperture or recess in a third wall of said third cylindrical element, and wherein after said bending said lip shaped portion engages with said aperture or recess.

Said lip shaped portion may be attached to said second wall via a beam having a beam axis, said lip shaped portion comprising a first part having a first width in direction substantially parallel to said beam axis and a second part having a second width in a direction substantially parallel to said beam axis, wherein said first part is arranged between said beam and said second part, and wherein said second width is smaller than said first width, wherein said second part engages with said aperture or recess of said third cylindrical element.

The method may further comprise releasing said at least one releasable attachment of said at least one tangential spacer after sliding said cylindrical elements into one another.

The various embodiments described above may be combined with one another. Embodiments described with respect to one aspect may be applied analogously with respect to one or more of the other aspects.

The cylindrical elements described above are, preferably, manufactured from a single cylindrical tube of any suitable material like stainless steel, cobalt-chromium, shape memory alloy such as Nitinol®, plastic, polymer, composites or other cuttable material. Alternatively, the cylindrical elements can be made by a 3D printing process. The thickness of that tube depends on its application. For medical applications the thickness may be in a range of 0.1-2.0 mm, preferably 0.1-1.0 mm, more preferably 0.1-0.5 mm, and most preferably 0.2-0.4 mm. The diameter of the cylindrical elements depends on its application. For medical applications the diameter may be in a range of 0.5-20 mm, preferably 0.5-10 mm, more preferably 0.5-6 mm. In many applications, the cylindrical element has a circular cross section. However, the term “cylindrical element” is not restricted to this interpretation. It may have an oval cross section or any other suitable cross section, including a rectangular cross section.

The slits and openings in all cylindrical elements to make, e.g., the at least one longitudinal element and the at least one tangential spacer can be made by laser cutting. The smaller slits which are made to just separate adjacent elements may have a width, preferably, in a range of 5-50 μm, more preferably 15-30 μm.

In this application, the terms “proximal” and “distal” are defined with respect to an operator, e.g. a robot or physician that operates the instrument or endoscope. For example, a proximal end part is to be construed as a part that is located near the robot or physician and a distal end part as a part located at a distance from the robot or physician, i.e., in the area of operation.

It is observed that the term “substantially” as used in the present document refers to manufacturing tolerances which exclude that features like sizes and distances have exact values. Depending on used manufacturing processes such tolerances may be smaller than 10%, preferably smaller than 5%, and even more preferably smaller than 1%.

### BRIEF DESCRIPTION OF THE DRAWINGS

Further features and advantages of the invention will become apparent from the description of the invention by way of non-limiting and non-exclusive embodiments. These embodiments are not to be construed as limiting the scope of protection. The person skilled in the art will realize that other alternatives and equivalent embodiments of the invention can be conceived and reduced to practice without departing from the scope of the present invention. Embodiments of the invention will be described with reference to the figures of the accompanying drawings, in which like or same reference symbols denote like, same or corresponding parts, and in which:

FIG. 1 shows a schematic perspective view of an invasive instrument assembly having two steerable instruments according to the prior art.

FIG. 2 shows a side view of a non-limiting embodiment of a steerable invasive instrument according to the prior art.

FIG. 3a shows a schematic picture of a colonoscopic instrument in use.

FIG. 3b shows a schematic picture of a gastroscopic instrument in use.

FIG. 4a provides a detailed perspective view of an embodiment of the elongated tubular body of a steerable instrument.

FIG. 4b provides a more detailed view of the distal end part of the elongated tubular body as shown in FIG. 4a.

FIG. 4c shows a longitudinal cross-sectional view of the elongated tubular

body of the steerable instrument as shown in FIG. 4a.

FIG. 4d shows a longitudinal cross-sectional view of the elongated tubular body of the steerable instrument as shown in FIG. 4a, wherein one proximal and one distal flexible zones are bent, thereby illustrating the operation of the steering arrangement.

FIG. 4e shows a longitudinal cross-sectional view of the elongated tubular body of the steerable instrument as shown in FIG. 4e, wherein additionally a second proximal and second distal flexible zones are bent, thereby further illustrating the operation of the steering arrangement.

FIG. 4f shows a longitudinal cross-sectional view of an embodiment of a steerable instrument having one proximal and one distal flexible zone.

FIG. 4g shows a perspective exploded view of the three cylindrical elements of the steerable instrument shown in FIG. 4f.

FIG. 4h shows a top view of an unrolled version of an embodiment of the intermediate cylindrical element of the steerable instrument shown in FIG. 4g. The intermediate cylindrical element can be formed by rolling the unrolled version into a cylindrical configuration and attaching adjacent sides of the rolled-up configuration by any known attaching means such as by a welding technique.

FIG. 4i shows a perspective view of a part of the elongated tubular body as shown in FIG. 4a, wherein the outer cylindrical element partially has been removed to show an embodiment of the longitudinal steering elements that have been obtained after providing longitudinal slits to the wall of an intermediate cylindrical element that interconnects the first proximal flexible zone and the first distal flexible zone of the elongated tubular body.

FIG. 5 shows a schematic cross-section of a further embodiment of a steerable invasive instrument, wherein the proximal end is provided with linear actuators for controlling bending of the distal end of the steerable instrument.

FIG. 6 shows a perspective exploded view of three cylindrical elements of a steerable instrument analogous to the embodiment of FIG. 4g, but with a varying diameter of the cylindrical elements.

FIG. 7 shows a schematic cross-section of a steerable instrument with cylindrical elements comparable to the embodiment as shown in FIG. 4d, but having different diameter.

FIGS. 8a and 8b show details of intermediate cylindrical elements of the prior art with fracture elements in perspective (8a) and side (8b) views, respectively.

FIGS. 9a, 9b and 9c show schematic drawings of an embodiment of a steerable instrument known from the prior art, in which inwardly bent lip shaped portions are used.

FIG. 10 shows a portion of the intermediate part of an intermediate

cylindrical element for a steerable instrument according to an embodiment of the invention.

FIGS. 11a and 11b show the intermediate cylindrical element of FIG. 10 coaxially aligned with an outer cylindrical element according to an embodiment.

FIG. 12 shows a portion an inner cylindrical element according to an embodiment.

FIGS. 13a, 13b show the assembly of FIGS. 11a, 11b coaxially aligned with the cylindrical element of FIG. 12.

FIGS. 14 shows an enlarged view of a tangential spacer according to an embodiment of the invention.

FIG. 15 shows a lip shaped portion for use as a radial spacer, as known from the prior art.

FIGS. 16 and 17 show embodiments of a lip shaped portion for use as a radial spacer according to embodiments of the invention.

FIGS. 18a, 18b and 18c show a portion of an outer cylindrical element provided with lip shaped portions according to an embodiment of the invention.

FIG. 19 shows a portion of an outer cylindrical element of provided with lip shaped portions forming radial spacers engaging with an inner cylindrical element according to an embodiment of the invention.

### DESCRIPTION OF EMBODIMENTS

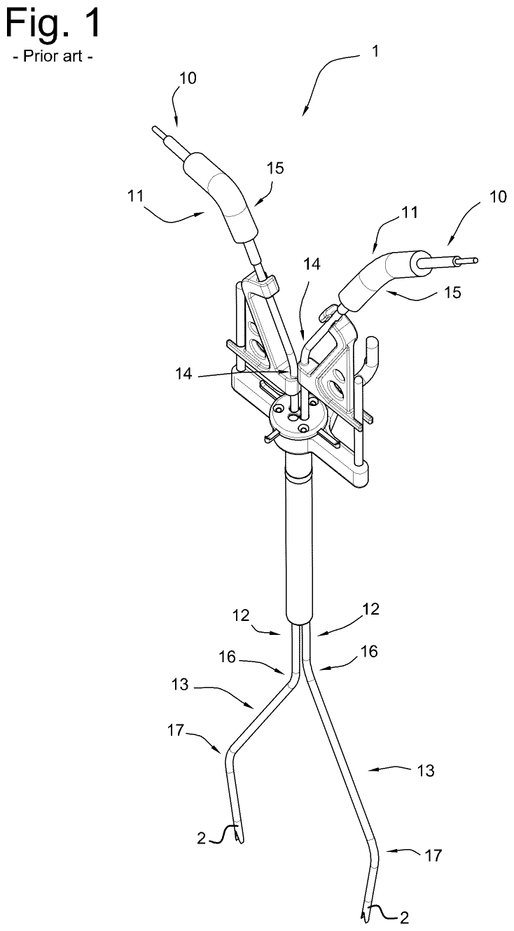

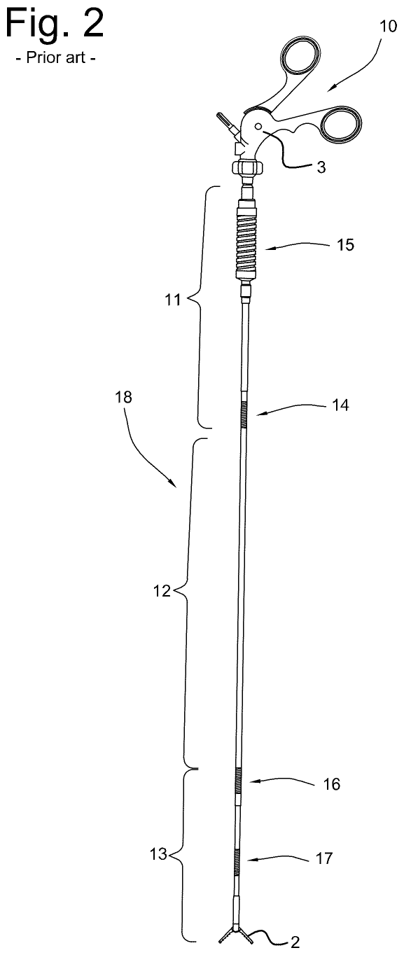

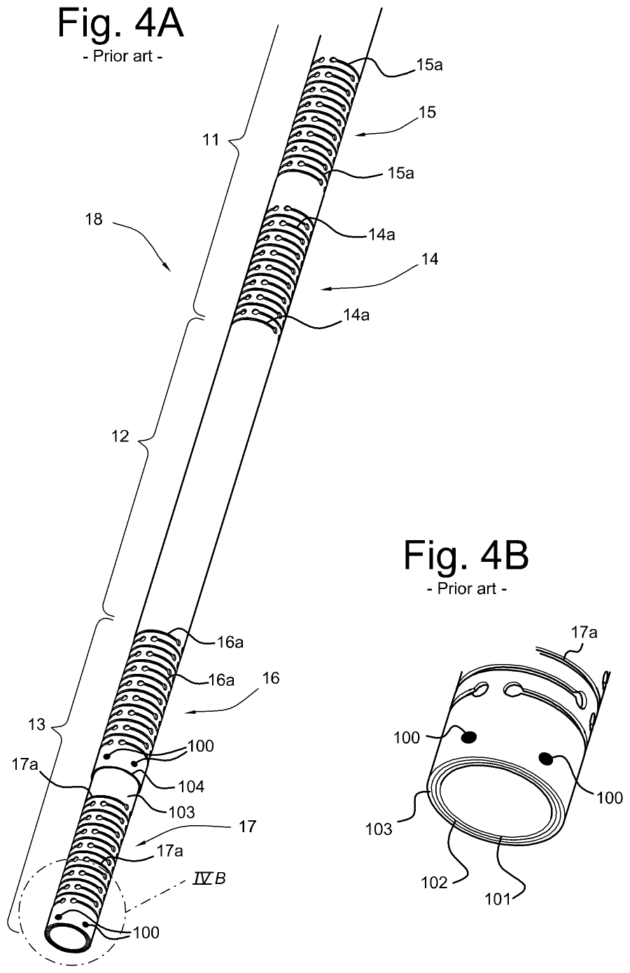

FIG. 1 shows a non-limiting embodiment of an invasive instrument assembly 1 having an introducer with two steerable invasive instruments 10. FIG. 2 shows a non-limiting embodiment of such steerable invasive instrument 10. Details of steerable invasive instruments 10 are explained in relation to FIGS. 4a to 4i.

FIG. 2 shows a side view of a steerable invasive instrument 10. The steerable instrument 10 comprises an elongated tubular body 18 having a proximal end part 11 including two actuation flexible zones 14, 15 (also referred to as flexible proximal zones), a distal end part 13 including two flexible distal zones 16, 17, and a rigid intermediate part 12. These flexible proximal zones 14, 15 are attached and/or connected to the flexible distal zones by suitable longitudinal elements (not shown in FIG. 2). By bending one such flexible proximal zone 14, 15, respectively, a corresponding flexible distal zone will also bend, as will be explained in detail hereinafter. The rigid intermediate part may also have one more bendable zones. However, these bendable zones are just flexible and their bending is not controlled by another bendable zone. If desired, more than two steerable flexible distal zones can be provided. At the distal end part 13 a tool, like a forceps 2 is arranged. At the proximal end part 11 a handle 3 is arranged that is adapted for opening and closing the jaw of the forceps 2 via, e.g., a suitable cable (not shown) arranged within the instrument. Cable arrangements for doing so are well known in the art.

While in the embodiment shown in FIG. 2, the intermediate part 12 is rigid, eventually comprising one or more bendable zones, which works well for many applications, in other applications, such as colonoscopy and gastroscopy, it is desired that the intermediate part 12 is flexible and/or bendable along substantially its whole length.

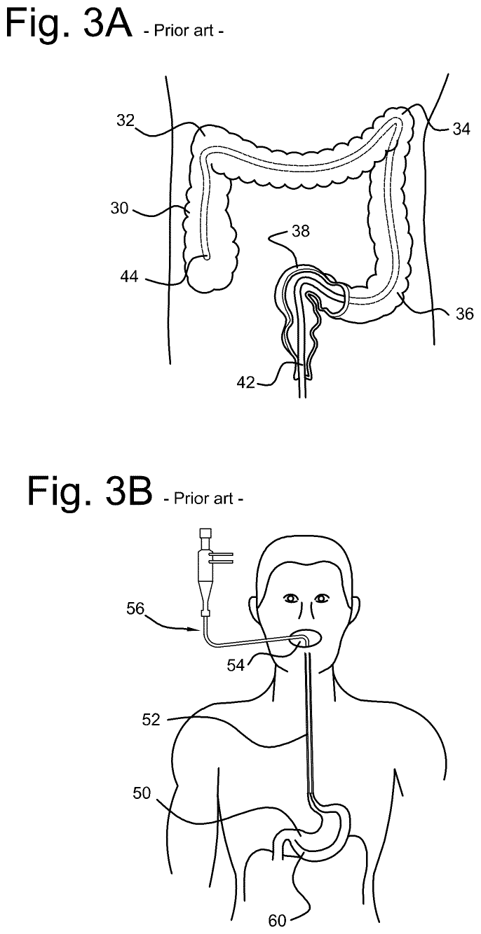

FIG. 3a shows a schematic view of a colonoscope 42 in use. The colonoscope 42 is inserted into a colon 30 of a human body. Typically, the colon 30 has several almost square angled sections 32, 34, 36, and 38. If a surgeon needs to operate an area of the colon 30 upstream from square angled section 32 the colonoscope 42 needs to be inserted into the colon 30 along a distance of up to 1.5 meter. Moreover, the colonoscope 42 needs to be so flexible that it can be guided from an anus through all squared angled sections 32-38 of the colon 30 easily without risks of damaging the inner wall of the colon 30.

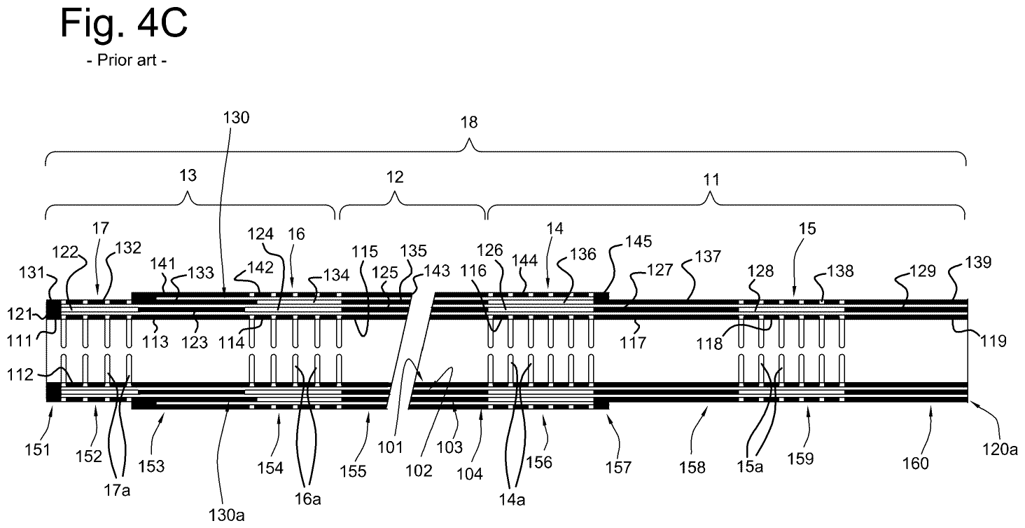

In operation, usually, several invasive instruments are inserted through the colonoscope 42 to provide one or more tools for some function at its distal end 44. In colonoscopy, such a tool typically includes a camera lens and a lighting element. To assist the surgeon in steering the camera view to the desired location and view in colon 30, typically, the distal end is deflectable from a longitudinal axis in all angular directions. This also holds for the inserted instruments with tools 2. That can be implemented by providing such an instruments with one or more flexible zones, like the flexible zones 16, 17 of the instrument shown in FIG. 2. These distal flexible zones are controlled by suitable steering cables, also referred to as longitudinal elements herein, accommodated in the instruments and connected and/or attached to a suitable steering mechanism at the proximal ends of the instruments. Such steering mechanism may, for example, be a steering mechanism as illustrated in FIG. 5, and/or may be controlled by a robot.

FIG. 3b shows a schematic view of a gastroscope 56 in use. The gastroscope 56 is inserted into a stomach 50 of a human body via mouth, oral cavity/throat 54 and oesophagus 52. Especially when a surgeon needs to operate a lower portion of the stomach 50, the gastronoscope 56 needs to be guided through several curved/angled sections. Therefore, the gastroscope 56 needs to be flexible such that there is little risk of damaging inner walls of the mouth/throat 54, oesophagus 52 and stomach 50.

In operation, usually, several invasive instruments are inserted through the gastroscope 56 to provide one or more tools for some function at its distal end 60. In gastroscopy, such a tool typically includes a camera lens and a lighting element. To assist the surgeon in steering the camera view to the desired location and direction in stomach 50, typically, the distal end 60 of the instrument is deflectable from a longitudinal axis in all angular directions. This also holds for the inserted instruments with tools 2. That can be implemented by providing such an instrument with one or more flexible zones, like the flexible zones 16, 17 of the instrument shown in FIG. 2. These flexible distal zones are controlled by suitable steering cables, also referred to as longitudinal elements herein, accommodated in the instruments connected to a suitable steering mechanism of these instruments.

The steerable instruments according to the invention can be used in such colonoscopes and gastroscopes. Therefore, general requirements to the presented instruments are that they show a high rotational stiffness, high longitudinal stiffness, flexibility along its entire length and deflectability at its flexible distal zones even in cases of long instruments, e.g., longer than 1 m, and with a relatively small diameter that fits to the working channels within or attached to colonoscopes and gastroscopes Also, such instruments should be designed such that they can be manufactured quite easily. In accordance with the invention this can be achieved with instruments having a tubular body with at least one tube element made from a metal and provided with suitable slotted structures to provide the instrument with enough flexibility along its entire length.

In FIGS. 4a to 4e embodiments are described of a steerable instrument comprising two flexible distal zones. It should however be understood that also other numbers of flexible distal zones are possible. Furthermore, in the embodiments shown in FIGS. 4a to 4i, the flexible distal zones are controlled, or actuated, via flexible proximal zones. It should however be understood that, alternatively, the flexible distal zones can be controlled by other means, such as by a robot, for example by a mechanism as illustrated in FIG. 5. The description relating to the intermediate and distal parts 12, 13 of the instruments apply analogously also for such other ways of controlling the one or more flexible distal zones.

FIG. 4a provides a detailed perspective view of the distal portion of the elongated tubular body 18 of the steerable instrument 10 and shows that the elongated tubular body 18 comprises of a number of co-axially arranged layers or cylindrical elements including an outer cylindrical element 104 that ends after the first flexible distal zone 16 at the distal end portion 13. The distal end portion 13 of the outer cylindrical element 104 is fixedly attached to the cylindrical element 103 located within and adjacent to the outer cylindrical element 104, e.g. by means of spot welding at welding spots 100. However, any other suitable attachment method can be used, including any mechanical snap fit connection or gluing by a suitable glue.

FIG. 4b provides a more detailed view of the distal end part 13 and shows that it includes three co-axially arranged layers or cylindrical elements being an inner cylindrical element 101, a first intermediate cylindrical element 102 and a second intermediate cylindrical element 103. The distal ends of inner cylindrical element 101, first intermediate cylindrical element 102 and second intermediate cylindrical element 103 are, preferably, all three fixedly attached to one another. This may be done by means of spot welding at welding spots 100. However, any other suitable attachment method can be used, including any mechanical snap fit connection or gluing by a suitable glue. The points of attachment may be at the end edges of inner cylindrical element 101, first intermediate cylindrical element 102 and second intermediate cylindrical element 103, as shown in the figures. However, these points of attachment may also be located some distance away from these edges, be it, preferably, between the end edges and the locations of the flexible zone 17.

It will be clear to the skilled person that the elongated tubular body 18 as shown in FIG. 4a comprises four cylindrical elements in total. The elongated tubular body 18 according to the embodiment shown in FIG. 4a comprises two intermediate cylindrical elements 102 and 103 in which the steering members, or longitudinal elements, of the steering arrangement are arranged. The steering arrangement in the exemplary embodiment of the elongated tubular body 18 as shown in FIG. 4a comprises the two flexible zones 14, 15 at the proximal end part 11 of the elongated tubular body 18, the two flexible zones 16, 17 at the distal end part 13 of the elongated tubular body 18 and the steering members that are arranged between related flexible zones at the proximal 11 and distal 13 end parts. An exemplary actual arrangement of the steering members is shown in FIG. 4c, which provides a schematic longitudinal cross-sectional view of the exemplary embodiment of the elongated tubular body 18 as shown in FIG. 4a.

FIG. 4c shows a cross section of the four layers or cylindrical elements mentioned above, i.e. the inner cylindrical element 101, the first intermediate cylindrical element 102, the second intermediate cylindrical element 103, and the outer cylindrical element 104.

The inner cylindrical element 101, as seen along its length from the distal end to the proximal end of the instrument, comprises a rigid ring 111, which is arranged at the distal end part 13 of the steerable instrument 10, a first flexible portion 112, a first intermediate rigid portion 113, a second flexible portion 114, a second intermediate rigid portion 115, a third flexible portion 116, a third intermediate rigid portion 117, a fourth flexible portion 118, and a rigid end portion 119, which is arranged at the proximal end portion 11 of the steerable instrument 10.

The first intermediate cylindrical element 102, as seen along its length from the distal end to the proximal end of the instrument, comprises a rigid ring 121, a first flexible portion 122, a first intermediate rigid portion 123, a second flexible portion 124, a second intermediate rigid portion 125, a third flexible portion 126, a third intermediate rigid portion 127, a fourth flexible portion 128, and a rigid end portion 129. The portions 122, 123, 124, 125, 126, 127 and 128 together form a longitudinal element 120 that can be moved in the longitudinal direction like a wire. The longitudinal dimensions of the rigid ring 121, the first flexible portion 122, the first intermediate rigid portion 123, the second flexible portion 124, the second intermediate rigid portion 125, the third flexible portion 126, the third intermediate rigid portion 127, the fourth flexible portion 128, and the rigid end portion 129 of the first intermediate element 102, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the inner cylindrical element 101, respectively, and are coinciding with these portions as well. In this description “approximately equal” means that respective same dimensions are equal within a margin of less than 10%, preferably less than 5%.

Similarly, the first intermediate cylindrical element 102 comprises one or more other longitudinal elements of which one is shown with reference number 120a.

The second intermediate cylindrical element 103, as seen along its length from the distal end to the proximal end of the instrument, comprises a first rigid ring 131, a first flexible portion 132, a second rigid ring 133, a second flexible portion 134, a first intermediate rigid portion 135, a first intermediate flexible portion 136, a second intermediate rigid portion 137, a second intermediate flexible portion 138, and a rigid end portion 139. The portions 133, 134, 135 and 136 together form a longitudinal element 130 that can be moved in the longitudinal direction like a wire. The longitudinal dimensions of the first rigid ring 131, the first flexible portion 132 together with the second rigid ring 133 and the second flexible portion 134, the first intermediate rigid portion 135, the first intermediate flexible portion 136, the second intermediate rigid portion 137, the second intermediate flexible portion 138, and the rigid end portion 139 of the second intermediate cylinder 103, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the first intermediate element 102, respectively, and are coinciding with these portions as well.

Similarly, the second intermediate cylindrical element 103 comprises one or more other longitudinal elements of which one is shown with reference number 130a.

The outer cylindrical element 104, as seen along its length from the distal end to the proximal end of the instrument, comprises a first rigid ring 141, a first flexible portion 142, a first intermediate rigid portion 143, a second flexible portion 144, and a second rigid ring 145. The longitudinal dimensions of the first flexible portion 142, the first intermediate rigid portion 143 and the second flexible portion 144 of the outer cylindrical element 104, respectively, are aligned with, and preferably approximately equal to the longitudinal dimension of the second flexible portion 134, the first intermediate rigid portion 135 and the first intermediate flexible portion 136 of the second intermediate element 103, respectively, and are coinciding with these portions as well. The rigid ring 141 has approximately the same length as the rigid ring 133 and is fixedly attached thereto, e.g. by spot welding or gluing. Preferably, the rigid ring 145 overlaps with the second intermediate rigid portion 137 only over a length that is required to make an adequate fixed attachment between the rigid ring 145 and the second intermediate rigid portion 137, respectively, e.g. by spot welding or gluing. The rigid rings 111, 121 and 131 are attached to each other, e.g., by spot welding or gluing. This may be done at the end edges thereof but also at a distance of these end edges.

In an embodiment, the same may apply to the rigid end portions 119, 129 and 139, which can be attached together as well in a comparable manner. However, the construction may be such that the diameter of the cylindrical elements at the proximal portion is larger, or smaller, with respect to the diameter at the distal portion. In such embodiment the construction at the proximal portion differs from the one shown in FIG. 4c. As a result of the increase or decrease in diameter an amplification or attenuation is achieved, i.e., the bending angle of a flexible zone at the distal portion will be larger or smaller than the bending angle of a corresponding flexible portion at the proximal portion.

The inner and outer diameters of the cylindrical elements 101, 102, 103, and 104 are chosen in such a way at a same location along the elongated tubular body 18 that the outer diameter of inner cylindrical element 101 is slightly less than the inner diameter of the first intermediate cylindrical element 102, the outer diameter of the first intermediate cylindrical element 102 is slightly less than the inner diameter of the second intermediate cylindrical element 103 and the outer diameter of the second intermediate cylindrical element 103 is slightly less than the inner diameter of the outer cylindrical element 104, in such a way that a sliding movement of the adjacent cylindrical elements with respect to each other is possible. The dimensioning should be such that a sliding fit is provided between adjacent elements. A clearance between adjacent elements may generally be in the order of 0.02 to 0.1 mm, but depends on the specific application and material used. The clearance preferably is smaller than a wall thickness of the longitudinal elements to prevent an overlapping configuration thereof. Restricting the clearance to about 30% to 40% of the wall thickness of the longitudinal elements is generally sufficient.

As can be seen in FIG. 4c, flexible zone 14 of the proximal end part 11 is attached to the flexible zone 16 of the distal end part 13 by portions 134, 135 and 136, of the second intermediate cylindrical element 103, which form a first set of longitudinal steering members of the steering arrangement of the steerable instrument 10. Furthermore, flexible zone 15 of the proximal end part 11 is attached to the flexible zone 17 of the distal end part 13 by portions 122, 123, 124, 125, 126, 127, and 128 of the first intermediate cylindrical element 102, which form a second set of longitudinal steering members of the steering arrangement. The use of the construction as described above allows the steerable instrument 10 to be used for double bending. The working principle of this construction will be explained with respect to the examples shown in FIGS. 4d and 4e.

For the sake of convenience, as shown in FIGS. 4c, 4d and 4e, the different portions of the cylindrical elements 101, 102, 103, and 104 have been grouped into zones 151-160 that are defined as follows. Zone 151 comprises the rigid rings 111, 121, and 131. Zone 152 comprises the portions 112, 122, and 132. Zone 153 comprises the rigid rings 133 and 141 and the portions 113 and 123. Zone 154 comprises the portions 114, 124, 134 and 142. Zone 155 comprises the portions 115, 125, 135 and 143. Zone 156 comprises the portions 116, 126, 136 and 144. Zone 157 comprises the rigid ring 145 and the parts of the portions 117, 127, and 137 coinciding therewith. Zone 158 comprises the parts of the portions 117, 127, and 137 outside zone 157. Zone 159 comprises the portions 118, 128 and 138. Finally, zone 160 comprises the rigid end portions 119, 129 and 139.

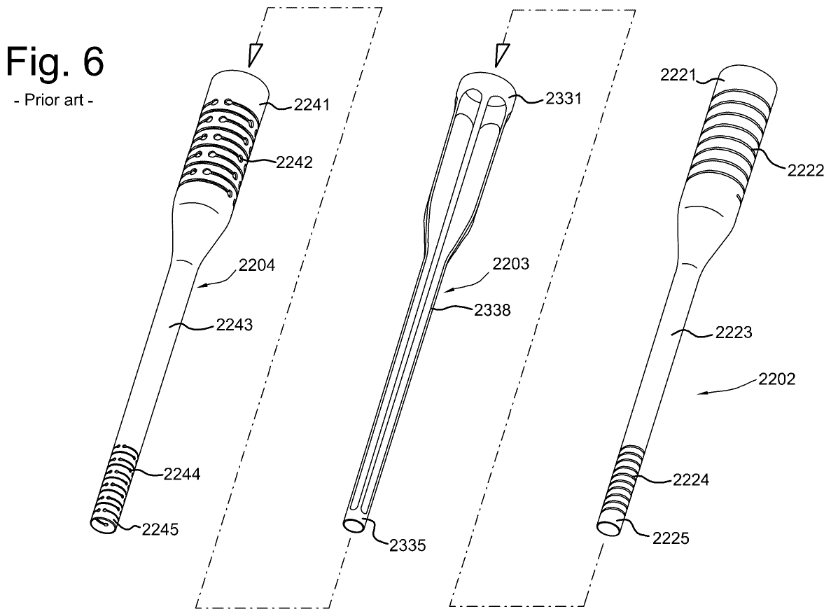

Although the portions 115, 125, 135 and 143 making up the zone 155 are described herein above as being rigid, alternatively, when the instrument is an instrument for use in e.g. colonoscopy or gastroscopy as described above, these portions will be flexible or bendable. However, contrary to the flexible distal portions, flexing or bending of the intermediate portions are not controlled by the operator of the instrument. Embodiments of such flexible intermediate portions are provided by the present invention, and are described in detail e.g. with respect to FIGS. 10-19 further below.

In order to deflect at least a part of the distal end part 13 of the steerable instrument 10, it is possible to apply a bending force, in any radial direction, to zone 158. According to the examples shown in FIGS. 4d and 4c, zone 158 is bent downwards with respect to zone 155. Consequently, zone 156 is bent downwards. Because of the first set of steering members comprising portions 134, 135, and 136 of the second intermediate cylindrical element 103 that are arranged between the second intermediate rigid portion 137 and the second rigid ring 133, the downward bending of zone 156 is transferred by a longitudinal displacement of the first set of steering members into an upward bending of zone 154 with respect to zone 155. This is shown in both FIGS. 4d and 4c.

It is to be noted that the exemplary downward bending of zone 156, only results in the upward bending of zone 154 at the distal end of the instrument as shown in FIG. 4d. Bending of zone 152 as a result of the bending of zone 156 is prevented by zone 153 that is arranged between zones 152 and 154. When subsequently a bending force, in any radial direction, is applied to the zone 160, zone 159 is also bent. As shown in FIG. 4c, zone 160 is bent in an upward direction with respect to its position shown in FIG. 4f. Consequently, zone 159 is bent in an upward direction. Because of the second set of steering members comprising portions 122, 123, 124, 125, 126, 127 and 128 of the first intermediate cylindrical element 102 that are arranged between the rigid ring 121 and the rigid end portion 129, the upward bending of zone 159 is transferred by a longitudinal displacement of the second set of steering members into a downward bending of zone 152 with respect to its position shown in FIG. 4d.

FIG. 4e further shows that the initial bending of the instrument in zone 154 as shown in FIG. 4d will be maintained because this bending is only governed by the bending of zone 156, whereas the bending of zone 152 is only governed by the bending of zone 159 as described above. Due to the fact that zones 152 and 154 are bendable independently with respect to each other, it is possible to give the distal end part 13 of the steerable instrument 10 a position and longitudinal axis direction that are independent from each other. In particular the distal end part 13 can assume an advantageous S-like shape. The skilled person will appreciate that the capability to independently bend zones 152 and 154 with respect to each other, significantly enhances the maneuverability of the distal end part 13 and therefore of the steerable instrument 10 as a whole.

Obviously, it is possible to vary the lengths of the flexible portions shown in FIGS. 4c to 4e as to accommodate specific requirements with regard to bending radii and total lengths of the distal end part 13 and the proximal end part 11 of the steerable instrument 10 or to accommodate amplification or attenuation ratios between bending of at least a part of the proximal end part 11 and at least a part of the distal end part 13.

The steering members comprise one or more sets of longitudinal elements that form integral parts of the one or more intermediate cylindrical elements 102, 103. Preferably, the longitudinal elements comprise remaining parts of the wall of an intermediate cylindrical element 102, 103 after the wall of the intermediate cylindrical element 102, 103 has been provided with longitudinal slits that define the remaining longitudinal steering elements.

Further details regarding the fabrication of the latter longitudinal steering elements are provided with reference to FIGS. 4f to 4h regarding an exemplary embodiment of a steerable instrument that comprises only one flexible zone at both its proximal 11 and distal end 13 parts.

FIG. 4f shows a longitudinal cross-section of a steerable instrument 2201 comprising three co-axially arranged cylindrical elements, i.e. inner cylindrical element 2202, intermediate cylindrical element 2203 and outer cylindrical element 2204. Suitable materials to be used for making the cylindrical elements 2202, 2203, and 2204 include stainless steel, cobalt-chromium, shape memory alloy such as Nitinol®, plastic, polymer, composites or other cuttable material. Alternatively, the cylindrical elements can be made by a 3D printing process.

The inner cylindrical element 2202 comprises a first rigid end part 2221, which is located at the distal end part 13 of the instrument 2201, a first flexible part 2222, an intermediate rigid part 2223, a second flexible part 2224 and a second rigid end part 2225, which is located at the proximal end part 11 of the instrument 2201.

The outer cylindrical element 2204 also comprises a first rigid end part 2241, a first flexible part 2242, an intermediate rigid part 2243, a second flexible part 2244 and a second rigid end part 2245. The lengths of the different parts of the cylindrical elements 2202 and 2204 are substantially the same so that when the inner cylindrical element 2202 is inserted into the outer cylindrical element 2204, the different parts are positioned against each other.

The intermediate cylindrical element 2203 also has a first rigid end part 2331 and a second rigid end part 2335 which in the assembled condition are located between the corresponding rigid parts 2221, 2241 and 2225, 2245 respectively of the two other cylindrical elements 2202, 2204. The intermediate part 2333 of the intermediate cylindrical element 2203 comprises three or more separate longitudinal elements which can have different forms and shapes as will be explained below. After assembly of the three cylindrical elements 2202, 2203 and 2204 whereby the element 2202 is inserted in the element 2203 and the two combined elements 2202, 2203 are inserted into the element 2204, at least the first rigid end part 2221 of the inner cylindrical element 2202, the first rigid end part 2331 of the intermediate cylindrical element 2203 and the first rigid end part 2241 of the outer cylindrical element 2204 at the distal end of the instrument are attached to each other. In the embodiment shown in FIGS. 4f and 4g, also the second rigid end part 2225 of the inner cylindrical element 2202, the second rigid end part 2335 of the intermediate cylindrical element 2203 and the second rigid end part 2245 of the outer cylindrical element 2204 at the proximal end of the instrument are attached to each other such that the three cylindrical elements 2202, 2203, 2204 form one integral unit.

Although the intermediate parts 2223, 2333 and 2243 are described herein above as being rigid, alternatively, when the instrument is an instrument for use in e.g. colonoscopy or gastroscopy as described above, these portions will be flexible or bendable. However, contrary to the flexible distal portions, flexing or bending of the intermediate parts are not controlled by the operator of the instrument. Embodiments of such flexible intermediate portions are provided by the present invention, and are described in detail e.g. with respect to FIGS. 10-19 further below.

In the embodiment shown in FIG. 4g the intermediate part 2333 of intermediate cylindrical element 2203 comprises a number of longitudinal elements 2338 with a uniform cross-section so that the intermediate part 2333 has the general shape and form as shown in the unrolled condition of the intermediate cylindrical element 2203 in FIG. 4h. From FIG. 4h it also becomes clear that the intermediate part 2333 is formed by a number of over the circumference of the intermediate cylindrical part 2203 equally spaced parallel longitudinal elements 2338. Advantageously, the number of longitudinal elements 2338 is at least three, so that the instrument 2201 becomes fully controllable in any direction, but any higher number is possible as well. Preferably, the number of longitudinal elements 2338 is 6 or 8.

The production of such an intermediate part is most conveniently done by injection molding or plating techniques or starting from a cylindrical tube with the desired inner and outer diameters and removing parts of the wall of the cylindrical tube required to end up with the desired shape of the intermediate cylindrical element 2203. However, alternatively, any 3D printing method can be used.

The removal of material can be done by means of different techniques such as laser cutting, photochemical etching, deep pressing, conventional chipping techniques such as drilling or milling, high pressure water jet cutting systems or any suitable material removing process available. Preferably, laser cutting is used as this allows for a very accurate and clean removal of material under reasonable economic conditions. The above mentioned processes are convenient ways as the member 2203 can be made so to say in one process, without requiring additional steps for attaching the different parts of the intermediate cylindrical element as required in the conventional instruments, where conventional steering cables must be connected in some way to the end parts. The same type of technology can be used for producing the inner and outer cylindrical elements 2202 and 2204 with their respective flexible parts 2222, 2224, 2242 and 2244.

FIG. 4i shows an exemplary embodiment of longitudinal (steering) elements 4 that have been obtained after providing longitudinal slits 5 to the wall of the second intermediate cylindrical element 103 that interconnects flexible proximal zone 14 and flexible distal zone 16 as described above. I.e., longitudinal steering elements 4 are, at least in part, spiraling about a longitudinal axis of the instrument such that an end portion of a respective steering element 4 at the proximal portion of the instrument is arranged at another angular orientation about the longitudinal axis than an end portion of the same longitudinal steering element 4 at the distal portion of the instrument. Were the longitudinal steering elements 4 arranged in a linear orientation, then a bending of the instrument at the proximal portion in a certain plane would result in a bending of the instrument at the distal portion in the same plane but in a 180 degrees opposite direction. This spiral construction of the longitudinal steering elements 4 allows for the effect that bending of the instrument at the proximal portion in a certain plane may result in a bending of the instrument at the distal portion in another plane, or in the same plane in the same direction. A preferred spiral construction is such that the end portion of a respective steering element 4 at the proximal portion of the instrument is arranged at an angularly shifted orientation of 180 degrees about the longitudinal axis relative to the end portion of the same longitudinal steering element 4 at the distal portion of the instrument. However, e.g. any other angularly shifted orientation, e.g. 90 degrees, is within the scope of this document. The slits are dimensioned such that movement of a longitudinal element is guided by adjacent longitudinal elements when provided in place in a steerable instrument.

The flexible portions 112, 132, 114, 142, 116, 144, 118, and 138 as shown in FIG. 4c, as well as the flexible parts 2222, 2224, 2242, and 2244 shown in FIGS. 4f and 4g can be obtained by the methods described in European patent application 08 004 373.0 filed on Oct. 3, 2008, page 5, lines 15-26, but any other suitable process can be used to make flexible portions.

Such flexible parts may have a structure as shown in FIGS. 4a and 4b. I.e., the flexibility may be obtained by a plurality of slits 14a, 15a, 16a, 17a. E.g., two circumferential slits may be provided in a cylindrical element along a same circumferential line where both slits are located at a certain distance from one another. A plurality of identical sets of circumferential slits 14a, 15a, 16a, 17a is provided at a plurality of distances in the longitudinal direction of the instrument, where consecutive sets are arranged at an angularly rotated position, e.g. each time 90 degrees rotated. In such an arrangement, all parts of the cylindrical element are still attached to each other.

Furthermore, if the portions 122, 123, 124, 125, 126, 127, and 128 of the first intermediate cylindrical element 102 and the portions 134, 135, and 136 of the second intermediate cylindrical element 103 that respectively form the first and second set of longitudinal steering members, as shown in FIG. 4c, are implemented as longitudinal steering elements 4 as shown in FIG. 4g, the fabrication methods described above can be used. The same applies to the longitudinal elements 2338 of FIGS. 4g and 4i. Moreover, any embodiment described in EP 2 762 058 A can be used according to the invention.

Otherwise, the longitudinal elements 4, 2338 can also be obtained by any other technique known in the art such as for example described in EP 1 708 609 A. The only restriction with respect to the construction of the longitudinal elements used in these portions is that the total flexibility of the instrument in these locations where the flexible portions coincide must be maintained.

The different co-axially arranged layers or cylindrical elements 101, 102, 103, 104, 2202, 2203 and 2204 as described above in relation to the exemplary embodiments of the steerable instruments shown in FIGS. 4c, 4d and 4d, respectively, may be produced by any of the known methods, provided that they are suitable to make a multilayer system. A multilayer system is to be understood as being a steerable instrument that comprises at least two separate sets of longitudinal elements 4, 2338 for transferring the movement of the proximal end part to the distal end part. The assembly of the different cylindrical elements can be realized in the same way as well. Preferred methods of producing the different cylindrical elements have been described in the above mentioned EP 2 762 058 A which is hereby incorporated by reference in its entirety.

In the above embodiments, the proximal portions and distal portions are constructed in a similar way. However, that need not always be the case as has been indicated above and as will become apparent hereinafter.

In the embodiment shown in FIG. 5 the distal end portion 13 is similar to the distal end portion 13 of the embodiment shown in e.g. FIGS. 4d and 4c, whereas the proximal end portion 11 has been modified. Around the proximal end portion 11′ there is provided a cylindrical housing 80 which is mounted on the external layer or outer cylindrical element 104 of the instrument. Furthermore, the external layer of the instrument at the proximal end portion is provided with a cylindrical member 83 such that between the zone 155 and the cylindrical member 83 a number of slits 67 are present. To the inner wall of the cylindrical housing 80 there are mounted two sets of linear actuators 81 and 82, respectively. A linear actuator is a device which can cause a translation movement of an element such as, for example, the longitudinal elements in this type of steerable instruments. Such linear actuators are generally known in the art and will not be described in more detail here, and they can be controlled by electronic devices such as computers.

The longitudinal elements of the second intermediate layer 103 are passing through the slits 67 and connected to the set 81 of linear actuators. The longitudinal elements of the first intermediate layer 102 are passing through the cylindrical member 83 and connected to the second set 82 of linear actuators. By means of an appropriate actuation of the linear actuators 81 and 82 the orientation of the flexible distal zones 152 and 154 can be changed so that the same effects are obtained as with the instrument according to FIGS. 4d and 4e. It is necessary that the actuation of the different linear actuators is done in a controlled manner as otherwise the change of orientation cannot be carried out. This means that if one actuator 81 is exerting a pulling force on its corresponding longitudinal element, the other actuators must be acting in a corresponding way, which means either exerting a smaller pulling force or exerting a pushing force so that the whole is in balance. The same applies if both sets of actuators are activated simultaneously. The areas of the instrument comprising the linear actuators are respective actuation zones of the instrument in the present embodiment.

Other robotic embodiments may be applied as well.

The steering arrangement at the proximal end may alternatively be

implemented by a ball shaped steering unit or a tiltable disc to which the longitudinal elements are attached. Such implementations are known as such. They may be operated by a human operator or by a robotic device which itself may be controlled by a human operator.

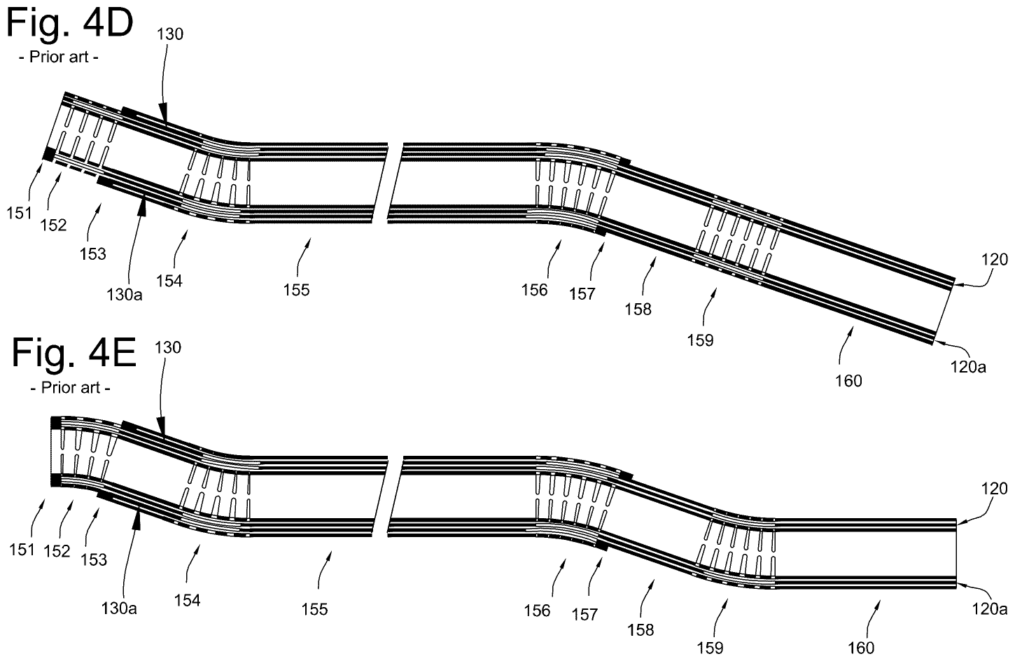

According to some embodiments, the proximal portion may have a wider diameter as shown in FIG. 6. The inner cylindrical element 2202 is composed of a first rigid end part 2225, a first flexible part 2224, an intermediate part 2223, a second flexible part 2222 and a second rigid end part 2221 which is normally used as the operating, or proximal, part of the instrument in that it serves to steer the other end of the unit. The outer cylindrical element 2204 is in the same way composed of a first rigid part 2245, a flexible part 2244, an intermediate part 2243, a second flexible part 2242 and a second rigid part 2241. The intermediate cylindrical element 2203 also has a first rigid end part 2335 and a second rigid end part 2331 which in the assembled condition are located between the corresponding rigid parts 2225, 2245 and 2221, 2241, respectively, of the two other cylindrical elements 2202, 2204. In the embodiment shown the longitudinal elements 2338 are of the type shown in FIG. 4g, but it will be obvious that any other type described above may be used as well. So far the construction is comparable to the instruments described above. The main difference with respect to the above embodiments is the use of a different set of diameters for some parts of the instrument. In the embodiment shown in FIG. 6 the parts 2222, 2221, 2331, 2242 and 2241 have a larger diameter than the other parts. In the parts 2223, 2338 and 2243 frusto-conical portions have been made in order to connect the small diameter parts with the large diameter parts. As shown in FIG. 6 the different parts can easily be assembled by inserting one into the other. The main reason, however, to have such an instrument with different diameters is that by using an operating part with a larger diameter, the movement of the other end is amplified, whereas if a smaller diameter is used the movement of the other end is reduced. Dependent of the application and its requirements larger diameters can be used to have the amplified movement or smaller diameters can be used to reduce the movement and increase accuracy.

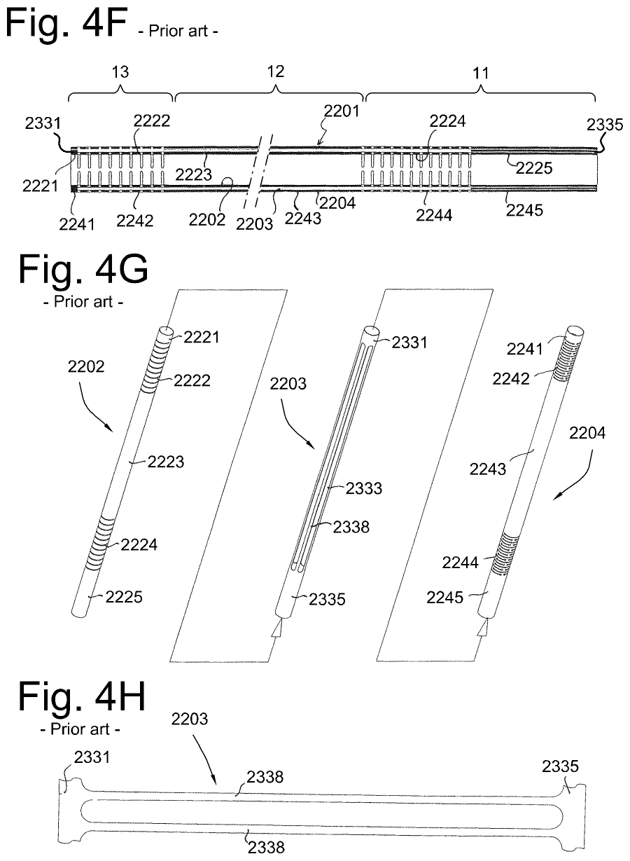

Such widening of the instrument with increasing diameter towards the proximal portions can also be applied in an instrument with more than two bendable portions, as shown in FIG. 7. In FIG. 7 there is shown an instrument having four layers and as such the instrument is comparable to the instrument of e.g. FIGS. 4d and 4c but the actuating portion of the cylindrical elements has a larger diameter compared to the handling end portion and in the zone 155 a frusto-conical part has been incorporated. As a result of the larger diameter of the actuating portion at the proximal end the movement of the handling portion at the distal end will be amplified upon bending, thereby amplifying the movement of the handling head. It is also possible to work in the opposite direction with a handling portion at the distal end with a larger diameter than the actuating portion at the proximal end, whereby the degree of movement is decreased, thereby improving accuracy of movement of the handling head.

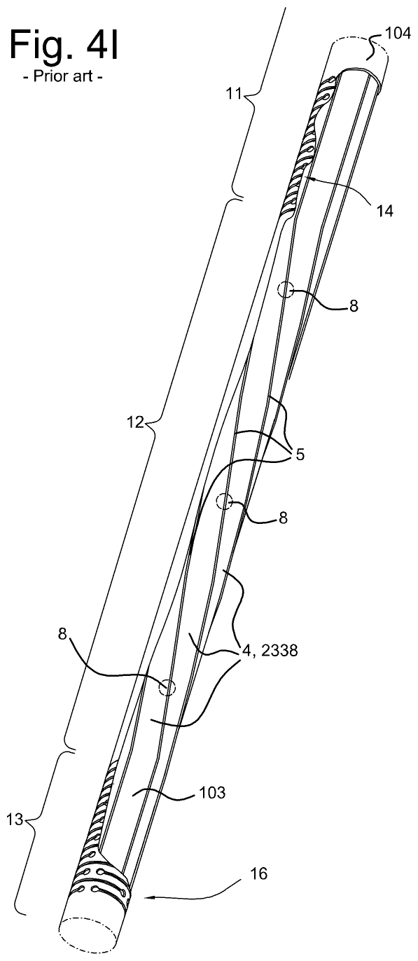

FIGS. 8a and 8b show an embodiment of a portion of an intermediate cylindrical element 102, 103 of the prior art, e.g. WO 2016/089202 A1, comprising fracture elements 7 having a circular shape arranged in an open space 6 formed between adjacent longitudinal elements 4. The fracture elements 7 are attached at ends of fracture locations 7.1 to respective adjacent longitudinal elements 4. These fracture elements keep the adjacent longitudinal elements together during assembly of the instrument, such as to maintain their relative ordering and geometrical coherency.

After assembly of the instrument, the fracture elements will be caused to break (fracture), due to a combination of bending forces and tension forces created by rolling of the circular shaped fracture element 7 along sides of the longitudinal elements 4. In general a fracture element 7 can be designed to have a shape such that one or more of shear forces, bending forces and tension forces act upon the fracture locations 7.1 of the fracture elements when adjacent longitudinal elements 4 are moved relative to one another.

In the embodiment of FIGS. 8a and 8b, the longitudinal elements 4 have thinner sections, creating the open space 6, and wider sections where they are only separated by a small slit 5. The reason is that in the steerable instruments having a rigid intermediate part 12, it was considered important that the longitudinal elements, along at least part of their length within the rigid intermediate part, be separated by only a small slit 5 such that movement of a longitudinal element would be guided by adjacent longitudinal elements, which provided for a well-controlled movement of the longitudinal elements. Also, the width of the longitudinal elements provide mechanical strength of the steerable instrument.

Since fracture or breakage of the fracture locations takes place after the different cylindrical elements have been slid into one another, the fracture elements will remain inside the instrument. As can be understood from inspection of FIGS. 8a and 8b, the shapes and dimensions of the open space 6 and the fracture element 7 will put limits on the relative movement of adjacent longitudinal elements 4. If at one of the fracture locations 7.1 fracture would accidentally not occur, the relative movement of adjacent longitudinal elements will be even more limited. Subsequent application of a bending force in excess of the range set by the dimensions of the open space 6 and the fracture element 7, may result in severe damage and even failure of the instrument. As can be understood from FIG. 8b, this will first cause the fracture element 7 to move into contact with one of the longitudinal elements 4 at an edge of the open space 6. If the applied force is sufficiently high, the fracture element 7 will be forced to move further, by sliding on top of or underneath the intermediate cylindrical element 102, 103, or into the slit 5. Here, the fracture element 7 might get stuck such as to hamper relative movements of elements within the instrument or otherwise causing severe damage to the instrument.

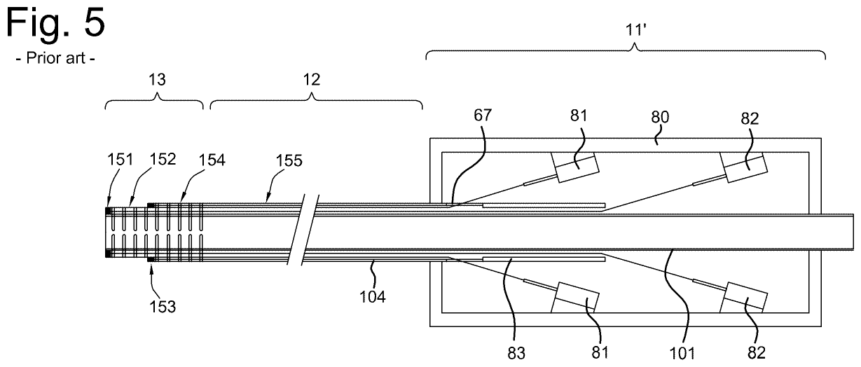

FIGS. 9a, 9b and 9c show an embodiment of a flexible zone of a steerable instrument as described in the prior art WO 2019/009710 A1. Radial spacers 294e are included to provide a distance between cylindrical elements 204, 208 such as to prevent clamping of longitudinal elements 282 of the intermediate cylindrical element 206 during bending of the flexible zones of the instrument.

As shown in FIG. 9a, the cylindrical element 204 is provided with lip shaped portions 294e in its wall 296.

As shown in FIG. 9b, in the intermediate cylindrical element 206, strip shape units forming a tangential spacer 275 are held together by a spacer portion 294f. At some locations, adjacent longitudinal element portions 282 are not spaced apart by spacers 275 but by a free space 320.

When the intermediate cylindrical element 206 is inserted into the cylindrical element 204, lip shaped portions 294e are located above a free space 320. Each lip shaped portion 294e is bent inwardly such that it remains in an inward bent position and extends through free space 320 and touches a portion of cylindrical element 208. FIG. 9c shows a cross section of the instalment in the longitudinal direction at a location of such a free space 320 when intermediate cylindrical elements 204, 206, 208 are inserted into one another.

Assuming that intermediate cylindrical element 206 has a height h1, then, lip shaped portion 294e is bent inwardly over a distance of h3 where h3>h1. Thus, at the location of the bent lip shaped portion 294e a well-defined radial space between intermediate cylindrical elements 204 and 208 is created which is larger than the height h1 of the longitudinal element portions 282. This supports movement of them in the longitudinal direction even in situations where the instrument is bent in the flexible zone.

However, the embodiment of FIGS. 9a-9c is associated with some drawbacks. If the lip shaped portions 294e, forming radial spacers, extends through an open space 320 between a tangential spacer 275 and the edges of longitudinal element portions 284, the lip shaped portion 294c may come into contact with the latter during bending of the flexible zone of the instrument, hence limiting the degree to which the instrument can be bent. When such open space 320 does not have a lip shaped portion 294e extending there through, the spacer portion 294f may come into contact with the edge of the longitudinal element portion 284, which may also limit the degree to which the instrument can be bent, and/or whereby the tangential spacer 275 may slide onto or below the longitudinal element portion 284, or force the adjacent longitudinal elements 276 apart, analogous to the problems described above with reference to FIGS. 8a, 8b.

Furthermore, the force applied for bending the lip shaped portion 294e has to be applied with a high degree of accuracy, i.e. as radial as possible, as otherwise the lip shaped portion may not be bent as intended. This might result in the lip shaped portion 294c not touching the cylindrical element 208, whereby the desired radial spacing may not be achieved, and/or in the lip shaped portion 294e abutting one or both of the adjacent longitudinal elements portions 282, causing friction during use of the instrument.

FIG. 10 shows an example of an intermediate portion 12 of an intermediate cylindrical element 1002, 1003 of a steerable instrument having a substantially flexible intermediate part according to an embodiment of the invention. The proximal end part of the intermediate cylindrical element may be a proximal end part as illustrated in FIGS. 4a, 4d, 4c, 4f, 4h, 4i, 6 and 7, or a proximal end part e.g. as illustrated in FIG. 5. The distal end part of the intermediate cylindrical element may be a distal end part as illustrated in FIGS. 4a to 4i and FIGS. 5 to 7. The features hereof have been discussed in detail above, and are therefore not repeated herein. The longitudinal elements 1038 shown in FIG. 10 are, at their respective ends, connected or attached to the longitudinal elements or portions thereof in the distal end part and in the proximal end part, respectively, or to longitudinal elements or portions thereof in the distal end part and to actuators located in the proximal end parts, in a manner as will be understood by the person skilled in the art. The longitudinal elements 1038 may be formed monolithically with the longitudinal elements in the proximal and distal end parts, respectively, or may be connected or attached thereto during manufacturing of the instrument.

The intermediate cylindrical element 1002, 1003 represents embodiments of the first cylindrical element described in the Summary section above.

In the embodiment shown in FIG. 10, the intermediate cylindrical element 1002, 1003 comprises a plurality of longitudinal elements 1038, extending throughout the intermediate part of the cylindrical element 1002, 1003. Via the longitudinal elements 1038, forces can be transferred from the proximal end part to the flexible distal zone such as to control bending of the distal end part of the steerable instrument, as described in detail above with reference to FIGS. 4 to 7. When more than one flexible distal zone are provided, these can be individually controlled in a manner as described above. The individual bending control of such flexible zones may be realized by the plurality of longitudinal elements 1038 divided into different sets of longitudinal elements, each set of longitudinal element arranged to control one flexible distal zone. Alternatively, one intermediate cylindrical element may be provided for each flexible distal zone.

As described above, e.g. with reference to FIG. 5, the longitudinal elements may, at the proximal end part, be connected to linear actuators, such that bending or flexing of the flexible distal zones of the instrument is controlled by a computer or robot. Alternatively, as also described above, the force may be applied to the longitudinal elements by bending or flexing of one or more proximal flexible zones. Ball shaped steering units or tiltable discs to which the longitudinal elements are attached may provided as alternative, possibly operated by a robotic instrument.

The longitudinal elements 1038 extend in a longitudinal direction 1008. This may be parallel to the cylindrical axis 1010, as in the embodiment of FIG. 10.

Alternatively, the longitudinal direction 1008 may be arranged in a helical manner with respect to the cylindrical axis 1010, e.g. as illustrated in FIG. 4i.

The longitudinal elements 1038 are arranged with a gap or open space 1006 between adjacent longitudinal elements 1038. In an embodiment, the longitudinal elements have uniform dimension throughout the intermediate portion and are uniformly distributed along the circumference of the cylindrical element, such that all open spaces 1006 have the same dimensions.

Tangential spacers 1007, 1017 are arranged in the open space 1006 between adjacent longitudinal elements 1038. The longitudinal elements 1038 and the tangential spacers 1007, 1017 are movable with respect to one another in the longitudinal direction.

Preferably, the longitudinal elements 1038 and the tangential spacers 1007, 1017 are not attached to one another, such as to not limit their respective movements in the longitudinal direction. The tangential spacers 1007, 1017 maintain a tangential distance between adjacent longitudinal elements 1038, while providing sufficient support of the longitudinal elements 1038 in the tangential direction, such as to provide mechanical strength and stability to the instrument.

Although FIG. 10 shows that tangential spacers 1007, 1017 are arranged to maintain a tangential distance between two adjacent longitudinal elements 1038, tangential spacers according to the invention can also be applied between one longitudinal element 1038 and another portion of cylindrical element 1002, 1003, where the longitudinal element 1038 can move in the longitudinal direction relative to that other portion of cylindrical element 1002, 1003. Then, tangential spacers 1007, 1017 are arranged to maintain a tangential distance between this longitudinal element 1038 and that other portion.

Although FIG. 10 illustrates an embodiment wherein a plurality of longitudinal elements 1038 are distributed around the circumference of the cylindrical element, this is not essential to the invention. In some embodiments, the intermediate cylindrical element 1002, 1003 comprises at least one longitudinal element 1038 (i.e., possibly only one longitudinal element), wherein an other portion of the cylindrical element extend in the longitudinal direction of the tube, substantially adjacent or next to the at least one longitudinal element, the other portion of the cylindrical element not itself forming a longitudinal element. Tangential spacers, i.e., at least one tangential spacer, are arranged tangentially adjacent the at least one longitudinal element, in an open space between the longitudinal element and the other portion of the cylindrical element.

In the embodiment of FIG. 10, a plurality of tangential spacers 1007 are arranged in the open space 1006. The number of tangential spacers 1007, their shapes and dimensions, and the distance between tangential spacers 1007 arranged in the same open space 1006 is determined e.g. by the desired mechanical properties of the intermediate cylindrical element 1002, 1003. Preferably, as shown in FIG. 10, the tangential spacers 1007 have an elongated shape, extending in the longitudinal direction 1008.

The longitudinal elements 1038 preferably have the same width w throughout their extension within the intermediate part. Thereby, the relative movement between the tangential spacers 1007, 1017 and the adjacent longitudinal elements 1038 will not be limited by widened portions of the longitudinal elements 1038. Thereby, the risk of a tangential spacer sliding on top of or underneath the longitudinal elements 1038 is reduced.

During assembly of the instrument, the tangential spacers 1007, 1017 may be attached to the adjacent longitudinal elements 1038 at fracture elements 1007a, 1017a which are fractured or destroyed after assembly of the instrument, by moving adjacent longitudinal elements 1038 relative to one another in opposite longitudinal directions as described in WO 2016/089202 A1 and PCT/NL2022/050318 of the present applicant. This provides stability of the intermediate cylindrical element and geometrical coherence of the longitudinal elements 1038 during assembly of the instrument.

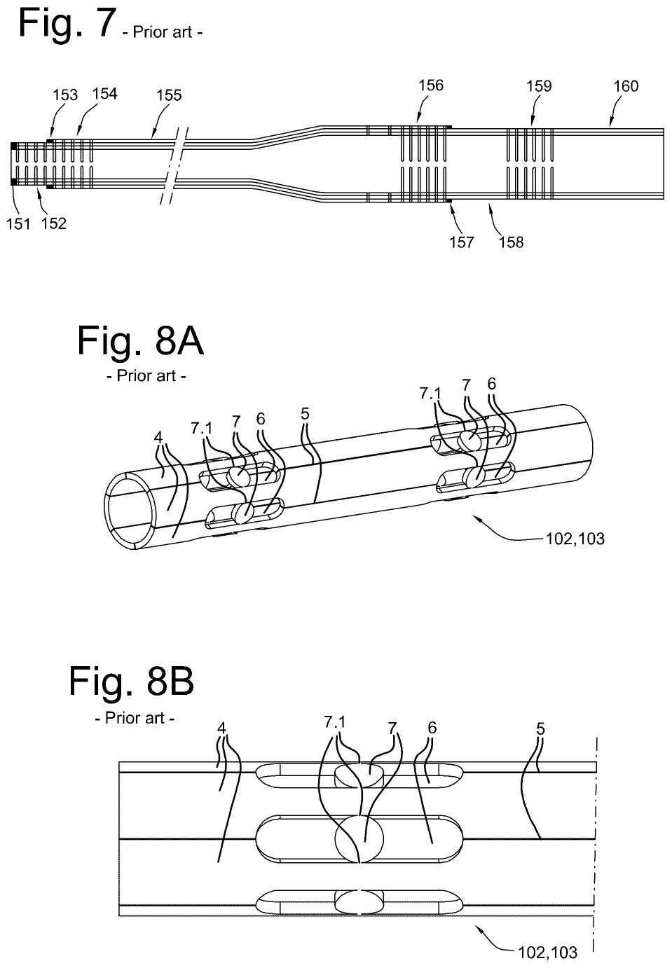

In the embodiment of FIG. 10, two different types of tangential spacers 1007, 1017 are provided. As can be seen, in every other open space 1006, as seen in the tangential direction, a first type of tangential spacers 1007 are arranged. As illustrated in FIGS. 11a and 11b, in an example, radial spacers 1094 are provided between each of these tangential spacers 1007. In the remaining every other open space 1006, a second type tangential spacer 1017 is arranged, which extends throughout substantially the whole length of the open space 1006 and which are not attached to at least one of the proximal and distal end.

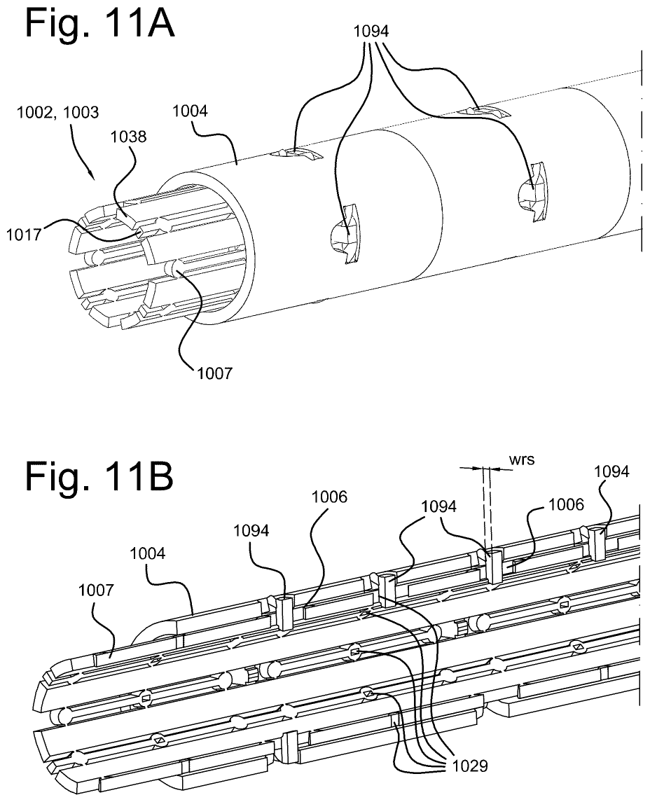

In accordance with the present invention, tangential spacer 1007 is provided with one or more apertures 1029. Also tangential spacer 1017 may be provided with one more of such apertures 1029. This is schematically indicated in FIG. 10 and shown in more detail for tangential spacer 1007 in FIG. 14.

FIGS. 11a, 11b show how lips 1094 bent inwardly from an outer cylindrical element 1004 into open spaces 1006 at longitudinal ends of tangential spacer 1007 and/or into the apertures 1029 of the tangential spacers 1007, 1017 can confine movement of the tangential spacers 1007, 1017.

FIG. 11a shows cylindrical element 1002, 1003 coaxially aligned with outer cylindrical element 1004. The outer cylindrical element 1004 represents embodiments of the second cylindrical element described in the Summary. In analogy to the intermediate cylindrical element 1002, 1003, the outer cylindrical element 1004 also comprises a proximal end part, a distal end part including at least one flexible distal portion, and an intermediate part between the proximal end part and the distal end part, which are aligned with the corresponding parts of the intermediate cylindrical element such as to realize a steerable instrument. Analogous to above, FIGS. 11a and 11b only show a portion of the intermediate part of the cylindrical elements.

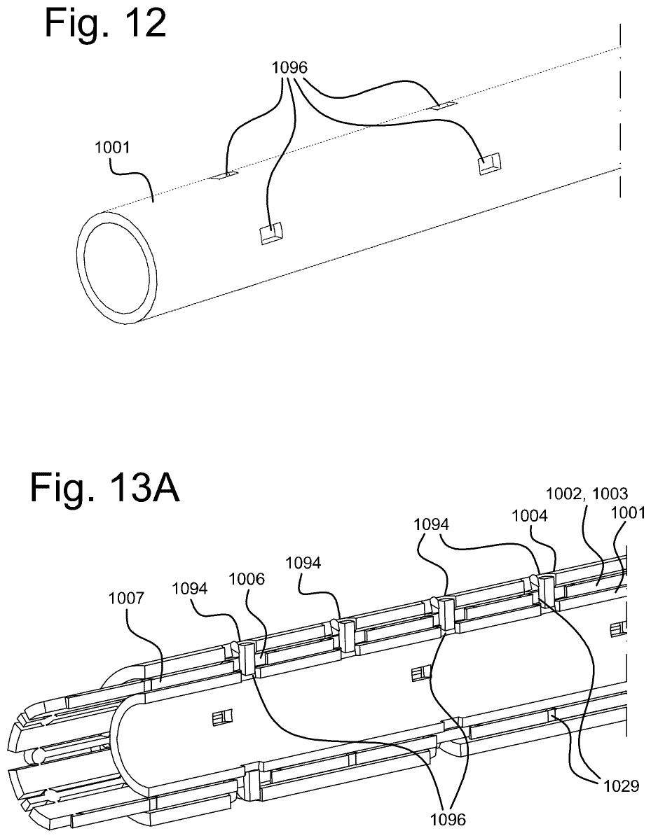

As can be seen, a plurality of radial spacers 1094 are provided, which are attached to, or formed from a part of, the outer cylindrical element 1004. The radial spacers 1094 extend in a radially inward direction, and protrude into, in fact extends through, the open space 1006 formed between adjacent longitudinal elements 1038 in the intermediate cylindrical element 1002, 1003. Preferably, the radial spacers 1094 are formed by lip shaped portions formed in the wall of the outer cylindrical element 1004, as will be described in more detail with respect to FIGS. 15-19.

FIG. 11b shows a cross section of FIG. 11a. Herein can be seen, that a radial spacer 1094 is provided at each longitudinal end of the tangential spacer 1007, and extends through the open space 1006.

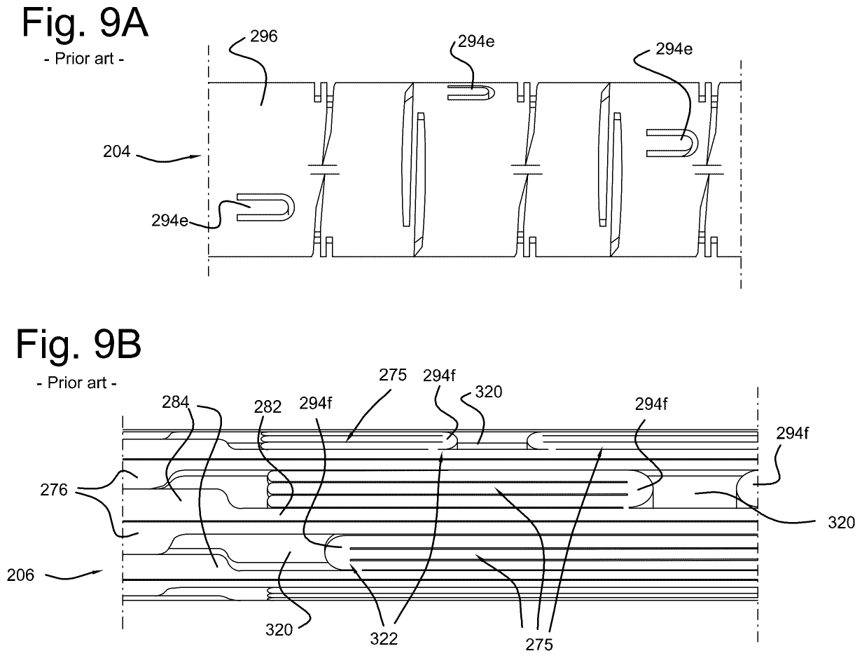

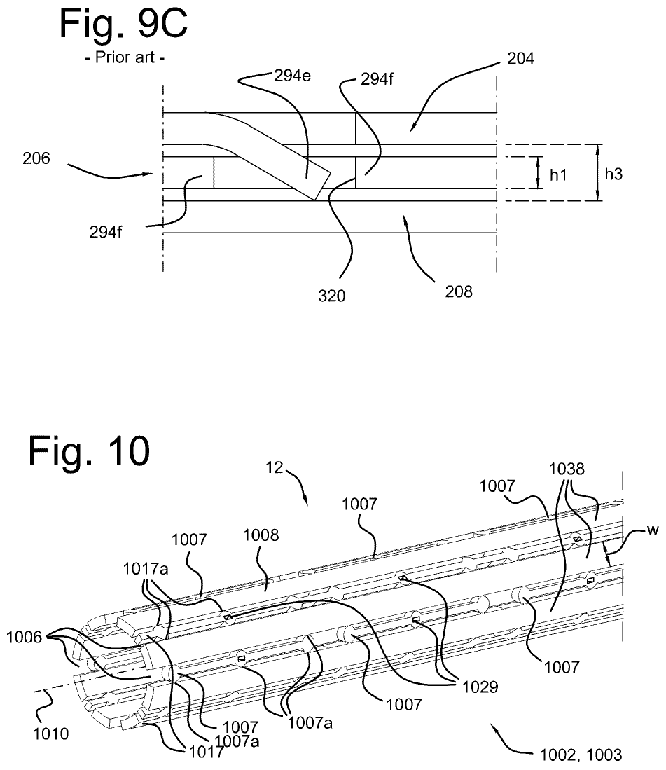

As will be described with reference to FIGS. 12 and 13, the radial spacers 1094 may set a distance between the outer cylindrical element 1004 and an inner cylindrical element 1001.

As can be understood from FIGS. 11a and 11b, the radial spacers 1094 confine, or limit, the movement of the tangential spacer 1007 in both directions along the extension of the longitudinal elements 1036, with respect to the second cylindrical element. The longitudinal elements 1038 can move relative to one another, enabling bending or flexing of the bendable part(s) of the cylindrical elements, without limitation or constrictions formed by tangential spacers 1007 coming into contact with edges of portions of the longitudinal elements 1038.

FIG. 12 shows an example of inner cylindrical element 1001. Similar to FIGS. 10, 11a and 11b, only the intermediate part of the inner cylindrical element 1001 is shown in FIG. 12. Analogous to the outer and intermediate cylindrical elements 1004, 1002, 1003, the inner cylindrical element 1001 comprises a proximal end part, a distal end part including at least one flexible distal portion, and an intermediate part extending between the proximal end part and the distal end part. In the assembled instrument, these different parts or portions are aligned with the corresponding parts or portions of the other cylindrical elements of the instrument. The inner cylindrical element 1001 may represent an embodiment of the third cylindrical element described in the Summary above.

The inner cylindrical element 1001 is provided with a plurality of apertures 1096, which after assembly of the instrument cooperate with the radial spacers 1094 such that the tips of the radial spacers 1094 extend in an aperture 1096, as illustrated in FIGS. 13a, 13b. The apertures 1096 are formed as through holes in the wall of the inner cylindrical element 1001, as this provides for easier manufacturing and a better degree of engagement or locking of the radial spacers 1094 in the apertures 1096. However, alternatively, they may be formed as recesses, forming a thinner portion of the wall of the inner cylindrical element.

FIGS. 13a, 13b show the inner cylindrical element 1001 slid into and coaxially aligned with the intermediate and outer cylindrical elements 1002, 1003, 1004 shown in FIGS. 11a, 11b. FIG. 13a shows a perspective view of a cut-through along the cylindrical axis, and FIG. 13b shows a cross section of the assembled inner, intermediate and outer cylindrical elements. It should be understood that although only one intermediate cylindrical element is indicated in FIGS. 11 and 13, more than one such intermediate cylindrical element may be provided, in analogy with the embodiments illustrated in FIGS. 4a to 4e.

In the embodiment shown in FIGS. 13a and 13b, the radial spacers 1094 protrude into the apertures 1096 formed in the inner cylindrical element 1001. By this arrangement the inner and outer cylindrical elements 1001, 1004 are fixed with respect to one another in axial, radial and tangential direction.

In alternative embodiments, the inner cylindrical element 1001 may be provided without apertures 1096, wherein the radial spacers 1094 are arranged to abut the inner surface of the wall of the inner cylindrical element 1001, and to, at least to some degree, provide fixation of the outer cylindrical element 1004 with respect to the inner cylindrical element 1001 by means of friction between the radial spacer 1094 and the inner cylindrical element 1001.

The radial spacers 1094 provide a distance between the inner cylindrical element 1001 and the outer cylindrical element 1004. The radial spacers 1094 have a radial dimension such that this distance is larger than the thickness of the longitudinal elements 1038 and the tangential spacers 1007, 1017 in the radial direction. Thereby, clamping or obstruction of the longitudinal elements 1038 between the inner and outer cylindrical elements 1001, 1004 during bending or flexing of the intermediate part of the instrument can be prevented.

In the embodiment of FIG. 13a, the tangential spacers 1007 are confined in a cage like structure formed by the radial spacers 1094, the inner cylindrical element 1001, the outer cylindrical element 1004, and the adjacent longitudinal elements 1038, without being connected or attached to any of these elements. Within this cage, the tangential spacer 1038 can move freely, and can be said to be floating.

FIG. 14 shows tangential spacer 1007, according to an embodiment of the present invention. The movement of this tangential spacer 1007, both in the longitudinal direction and in the tangential direction of the instrument, is confined by one or more radial spacers 1094 described above. The tangential spacer 1007 may be freely floating—i.e., is not attached or connected to an other portion of cylindrical element 1002, 1003—at one or both of its longitudinal ends. Tangential spacer 1007 is provided with an aperture 1029 which, in an embodiment, has a predetermined length in the longitudinal direction and a predetermined width, wₐ, in the tangential direction. In the assembled instrument, a radial spacer 1094 protrudes through the aperture 1029. Thereby, both in the longitudinal direction and tangential direction, the movement of the tangential spacer 1007 with respect to the outer cylindrical element 1004 is limited by one radial spacer 1094, which protrudes through the aperture 1029. The radial spacer 1094 may have a radial spacer width, wᵣₛ, in its tangential direction in a range of 90%<wᵣₛ<100%, preferably 95%<wᵣₛ<100%, and even more preferred 98%<wᵣₛ<100% of the aperture width, wₐ, of aperture 1029, as seen in a tangential direction when the tangential spacer 1007 is arranged in the instrument. The radial spacer width, wᵣₛ, may be a width as indicated in FIG. 15-17 described further herein below. Then, tangential spacer 1007 can only move with a small, well defined maximum amount in the tangential direction relative to cylindrical element 1004. However, alternatively, radial spacer 1094 may have a radial spacer width wᵣₛ equal to or even slightly larger—e.g. by up to 5%—than the aperture width wₐ of aperture 1029 such that when radial spacer 1094 is bent into aperture 1029 it clamps tangential spacer 1007. It is observed that, because of the tangential confinement in movement of tangential spacer 1007, also the other parts of cylindrical element 1002, 1003, including the one or more longitudinal elements 1038, are confined in their tangential movement relative to cylindrical element 1004. The radial spacer 1094 may be, but need not be, in contact with cylindrical element 1001. E.g., the number of radial spacers 1094 and the corresponding, optional, apertures 1096 of the inner cylindrical element 1001 may be adapted to the number of tangential spacers 1007. Tangential spacers 1007 may be provided with more than one aperture 1029 through which a radial spacer 1094 protrudes.

In an embodiment, tangential spacers 1017 may also be provided with one or more apertures 1029, through which radial spacers 1094 extend, in the same way as explained with reference to FIG. 14, and having the same technical effects. Moreover, the embodiment of FIG. 14 may be combined with the one of FIGS. 11A-13B, such that lips 1094 extending inside apertures 1029 confine longitudinal and tangential movement of tangential spacers 1007 and/or 1017, and lips 1049 extending inside spaces 1006 maintain, e.g., at least a radial distance between cylindrical elements 1001 and 1004.

FIGS. 15 to 17 show different embodiments of a lip shaped portion 1097, 1098, 1099 formed in the wall of a cylindrical element, for use as a radial spacer, such as the radial spacer 1094 described above. The lip shaped portion may be formed by laser cutting one or more slots in the wall of the cylindrical element. The resulting radial spacer preferably is flexible or at least has some degree of flexibility or elasticity with respect to the cylindrical element from which it is formed.

Advantageously, the lip shaped portions 1097, 1098, 1099 are formed in the outer cylindrical element 1004, whereby the radial spacers can be formed by bending each of these lip shaped portions in a radially inward direction after assembly of the instrument. Alternatively, it can be formed in the inner cylindrical element 1001, and be bent in a radially outward direction in order to form radial spacers.

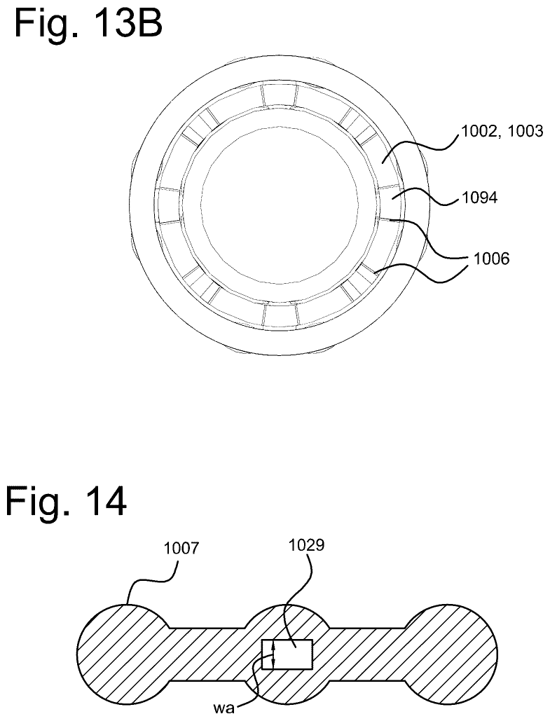

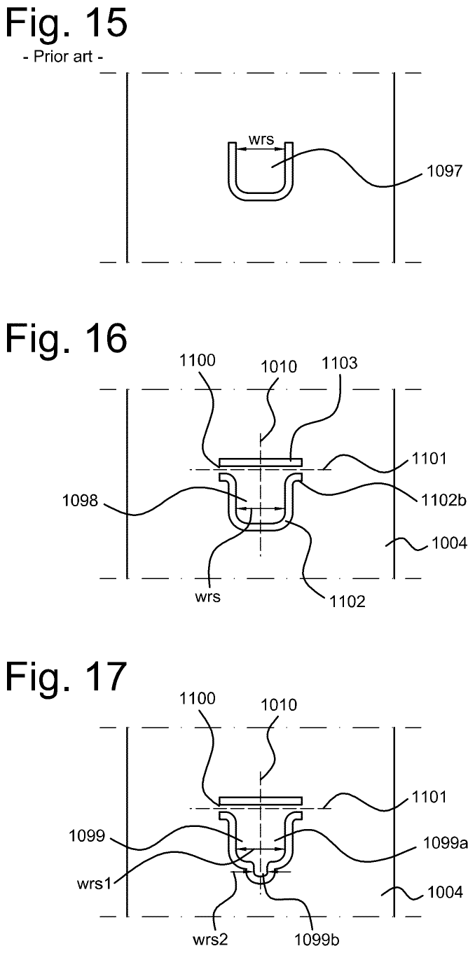

In addition to forming the radial spacers 1094 used in the intermediate part 12 of the instrument, the lip shaped portions 1098, 1099 shown in FIGS. 16 and 17 may be used to form radial spacers provided in the distal flexible zones 16, 17 and, if present, in proximal flexible zones 14, 15, of the instrument, thereby replacing the lip shaped portions 294e of FIGS. 9a to 9c.

The lip shaped portion 1097 shown in FIG. 15 substantially corresponds to the lip shaped portion 294f shown in FIGS. 9a to 9c.

FIGS. 16 and 17 show lip shaped portions 1098, 1099 according to embodiments of the invention, representing further developments of the lip shaped portion of the prior art.

As shown in FIGS. 16 and 17, the lip shaped portion 1098, 1099 is attached to, or forms a connection with, the wall of the second cylindrical element 1004 via a beam 1100 having a beam axis 1101.

In the embodiment shown in FIGS. 16 and 17, the beam axis 1101 extends in a direction substantially perpendicular to the cylindrical axis 1010 (longitudinal direction) of the second cylindrical element 1004. This is advantageous for embodiments of the steerable instrument where, in the intermediate region, the longitudinal elements 1038 extend in a direction parallel to the cylindrical axis, i.e., the longitudinal elements 1038 having their longitudinal direction 1008 along the cylindrical axis.

Alternatively, the beam axis 1101 may be oriented at a different angle with respect to the cylindrical axis 1010, for instance, depending on the direction of extension of the longitudinal elements 1038 throughout the intermediate region. As discussed above, in some embodiments the longitudinal elements may be arranged to form, at least partly, a helix, or be arranged to extend in an at least partly spiraling direction, with respect to the cylindrical axis. In such embodiments, the angle may be slightly less than 90 degrees. It is observed, however, that also in case the longitudinal elements 1038 extend along a helical path, the beam axes 1101 may extend perpendicular to the cylindrical axis 1010.

In short, the beam axis 1101 may extend in a direction substantially perpendicular to the direction of extension of the longitudinal elements 1038, at the location of the beam axis, in the intermediate cylindrical element together with which the outer cylindrical element 1004 is intended to be used.

The lip shaped portion 1098, 1099 is defined by a first slot 1102 formed in the wall of the cylindrical element 1004. This slot 1102 extends around the lip shaped portion 1098, 1099 on all sides, except for where the lip shaped portion is attached to the wall via the beam 1100. In addition, first slot portions 1102b extend in a direction substantially parallel to the beam axis 1101 on either side of the lip shaped portion 1098, 1099, at one side of the beam 1100. The beam 1100 is defined by a second slot 1103 provided in the wall of the second 1004, wherein the second slot 1103 extends in a direction substantially parallel to the beam axis 1101 at the other side of the beam 1100.

The slots 1102, 1103 may advantageously be formed by laser cutting through the wall of a cylindrical element.

By this construction of the lip shaped portion 1098, 1099 and the beam 1100 forming the attachment to the cylindrical element 1004, defined by the slots 1102, 1003, bending of the lip shaped portion in the radial direction of the cylindrical element is defined with high accuracy. If a force is applied in the radial direction onto the lip shaped portion, this will bend around the beam axis 1101.

In the embodiment shown in FIG. 17, the lip shaped portion 1099 comprises a first part 1099a having a first width in a direction substantially parallel to the beam axis 1101 and a second part 1099b having a second width, which is smaller than the first width. The first part 1099a is located closest to the beam 1100 and forms an attachment thereto, and the second part 1099b forms an outer tip of the lip shaped portion 1099. The second part 1099b is advantageously used to engage with, e.g. to be inserted into, an aperture or recess 1096 of a further, e.g. inner, cylindrical element 1001, arranged coaxial with the outer cylindrical element 1004, as illustrated in FIGS. 13a, 13b.

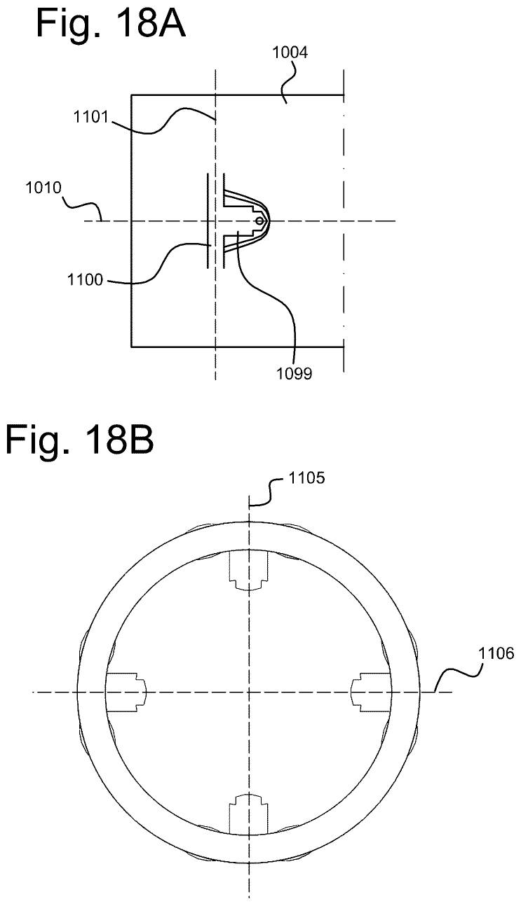

FIGS. 18a to 18c show an embodiment of a portion of an outer cylindrical element 1004 provided with a plurality of lip shaped portions 1099 according to the embodiment shown in FIG. 17. In the embodiment of FIGS. 18a to 18c, the beam axis 1101 is oriented perpendicular to the cylinder axis 1010, however, as described above, the orientation will be set dependent on the orientation of the longitudinal elements in the intermediate region. FIG. 18a illustrates the lip shaped portion prior to bending. As can be seen in this figure, in this embodiment, the beam 1100 is symmetrical with respect to the beam axis 1101 and the lip shaped portion is symmetrical with respect to the cylinder axis 1010. In other embodiments, as discussed above, the beam axis may be oriented at a different angle, non-perpendicular, to the cylinder axis 1010. In this case, the lip shaped portion is symmetrical with respect to an axis extending perpendicular to the beam axis.

As shown in FIGS. 18b and 18c, the lip shaped portions 1099 is bent in the radial inward direction such as to form radial spacers 1094. As illustrated in FIG. 18b, bending of the lip shaped portion will result in a straight and axisymmetric radial spacer. As can be seen, the radial spacers are symmetric with respect to radial axes 1105, 1106.

It should be noted that the number and distribution of the lip shaped portions and radial spacers illustrated in FIGS. 18a to 18c is merely an example provided for illustration, and that the number of lip shaped portions/radial spacers, and their distribution around the circumference of the cylindrical element and along the axial direction of the cylindrical element, is set in accordance to various properties to be realized in the assembled steerable instrument, such as the flexibility and/or mechanical strength of the intermediate region.

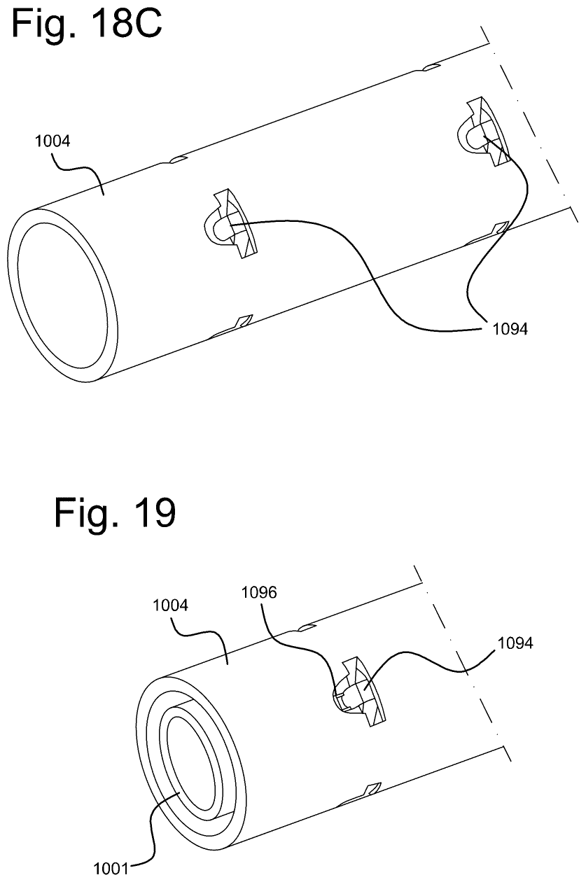

FIG. 19 shows a portion of an outer cylindrical element 1004 in which a lip shaped portion is provided, assembled with an inner cylindrical element 1001. The radial spacer 1094 engages with an aperture 1096 provided in the inner cylindrical element 1001. For clarity of illustration of the principle of locking the inner and outer cylindrical elements with respect to one another, the one or more intermediate cylindrical elements 1002, 1003 have been left out in FIG. 19.

Although with reference to the embodiments illustrated in FIGS. 18 and 19, the lip shaped portion forming the radial spacer is described as being formed in an outer cylindrical element 1004, this need not be the case, but the lip shaped portions forming the radial spacers may alternatively be formed in an inner cylindrical element 1001, wherein the radial spacers are subsequently formed by bending the lip shaped portions in a radially outward direction, such as to engage with the outer cylindrical element 1004.

The steerable instrument may be formed by providing an inner cylindrical element 1001, an outer cylindrical element 1004, and one or more intermediate cylindrical elements 1002, 1003 as described herein above. Thereby, an inner cylindrical element 1001 as illustrated in FIG. 12, one or more intermediate cylindrical elements 1002, 1003 as illustrated in FIG. 10, and an outer cylindrical element as illustrated in FIGS. 11a, 11b, 18a, 18b and 18c, may be provided.

Subsequently, these cylindrical elements 1001-1004 are coaxially aligned by sliding them into one another, and aligning them along the direction of the cylinder axis such that corresponding flexible and bendable parts and regions of the various cylindrical elements are aligned. Subsequently, the lip shaped portions 1094 formed in the outer cylindrical element is bent radially inwards by the application of a force applied perpendicular to the lip shaped portion in the wall, such that the lip shaped portion bends radially inwards until it protrudes through the open space 1006 or opening 1029 (FIG. 14) and, optionally, engages with the aperture 1096 provided in the wall of the inner cylindrical element 1001.

After assembly of the instrument, the releasable attachments of the tangential spacers 1007, 1017 are released. The releasable attachments are preferably provided by fracture elements 1007a, 1017a, which are fractured by bending of the instrument in various directions after assembly thereof. Alternatively, such fracture elements may be destroyed by an energy, e.g. laser, beam or by applying fatigue.

By providing cylindrical elements having an intermediate portion in accordance with one or more of the embodiments described herein above with reference to FIGS. 10 to 19, a steerable instrument having a flexible intermediate part 12, and which is thereby flexible substantially throughout its extension, for at least as far as the instrument will be inserted into a body, can be realized. In particular, thereby a steerable instrument suitable for applications such as colonoscopic and/or gastroscopic examination and/or surgery is provided.

It will be clear to a person skilled in the art that the scope of the invention is not limited to the examples discussed in the foregoing, but that several amendments and modifications thereof are possible without deviating from the scope of the invention as defined in the attached claims. While the invention has been illustrated and described in detail in the figures and the description, such illustration and description are to be considered illustrative or exemplary only, and not restrictive. The present invention is not limited to the disclosed embodiments but comprises any combination of the disclosed embodiments that can come to an advantage.

Variations to the disclosed embodiments can be understood and effected by a person skilled in the art in practicing the claimed invention, from a study of the figures, the description and the attached claims. In the description and claims, the word “comprising” does not exclude other elements, and the indefinite article “a” or “an” does not exclude a plurality. In fact it is to be construed as meaning “at least one”. The mere fact that certain features are recited in mutually different dependent claims does not indicate that a combination of these features cannot be used to advantage. Any reference signs in the claims should not be construed as limiting the scope of the invention. Features of the above described embodiments and aspects can be combined unless their combining results in evident technical conflicts.

1. A steerable instrument for endoscopic and/or invasive type of applications, such as in surgery, the instrument comprising:
a first cylindrical element comprising a first proximal end part, a first distal end part including at least one first flexible distal portion, and a first intermediate part between said first proximal end part and said first distal end part, and at least one longitudinal element arranged for transferring a force from said first proximal end part to said at least one first flexible distal portion and thereby control bending of said at least one first flexible distal portion;
at least one tangential spacer arranged tangentially adjacent to said at least one longitudinal element in an open space between said at least one longitudinal element and an other portion of the first cylindrical element;
a second cylindrical element arranged coaxially with said first cylindrical element, and comprising a second proximal end part, a second distal end part including at least one second flexible distal portion, and a second intermediate part between said second proximal end part and said second distal end part, corresponding to and substantially aligned with said first proximal end part, said first distal end part and said first intermediate part of said first cylindrical element, respectively;
at least one radial spacer attached to the second cylindrical element and protruding into an aperture in the tangential spacer;
such that a movement of the tangential spacer both in a longitudinal direction and in a tangential direction of the steerable instrument is confined by said at least one radial spacer, and optionally
at least one further radial spacer attached to the second cylindrical element and protruding in the open space at a longitudinal end of the at least one tangential spacer.

2. The steerable instrument according to claim 1, wherein the first cylindrical element is a first cylindrical tube, and both the at least one longitudinal element and the at least one tangential spacer are portions of the first cylindrical tube.

3. The steerable instrument according to claim 1, wherein the second cylindrical element is a second cylindrical tube.

4. The steerable instrument according to claim 3, wherein said at least one radial spacer and said at least one further radial spacer are formed by a lip shaped portion of the second cylindrical tube and protruding from the second cylindrical tube.

5. The steerable instrument according to claim 4, wherein the lip shaped portion has a lip shaped portion width and the aperture has an aperture width wherein the lip shaped portion width is either smaller than the aperture width or is configured such that it clamps the tangential spacer when bent into the aperture.

6. The steerable instrument according to claim 4,
wherein said lip shaped portion is formed in a wall of the second cylindrical tube and attached thereto via a beam having a beam axis extending substantially perpendicular to said longitudinal direction;
wherein said lip shaped portion is defined by a first slot formed in said wall, said first slot extending around said lip shaped portion except for where the lip shaped portion is attached to said beam;
said first slot further extending in a direction substantially parallel to said beam axis on either side of said lip shaped portion at one side of said beam; and
wherein said beam is further defined by a second slot provided in said wall in a direction substantially parallel to said beam axis at another side of said beam.

7. The steerable instrument according to claim 1, wherein in said first intermediate part said at least one longitudinal element has a length in the longitudinal direction and a width in a direction substantially perpendicular to said longitudinal direction, wherein said width is substantially constant throughout said length.

8. The steerable instrument according to claim 1, further comprising a third cylindrical element arranged coaxially with said first cylindrical element and said second cylindrical element,
wherein said first cylindrical element is arranged between said second cylindrical element and said third cylindrical element, and
wherein at least one of the following applies: said at least one radial spacer or said at least one further radial spacer is arranged to abut said third cylindrical element, is engaged with said third cylindrical element, and is attached to said third cylindrical element.

9. The steerable instrument according to claim 8, wherein said third cylindrical element (is a third cylindrical tube provided with at least one recess or aperture, and wherein said at least one radial spacer or said at least one further radial spacer protrudes into said at least one recess or aperture.

10. The steerable instrument according to claim 9, wherein said radial spacer or said at least one further radial spacer comprises a first part having a first width and a second part having a second width which is smaller than said first width, and wherein said second part protrudes into said recess or aperture.

11. The steerable instrument according to claim 8, wherein said at least one radial spacer or said at least one further radial spacer forms a distance between said second cylindrical element and said third cylindrical element in a radial direction which is larger than a thickness of said at least one longitudinal element and said at least one tangential spacer in said radial direction.

12. The steerable instrument according to claim 8, wherein said third cylindrical element comprises a third proximal end part, a third distal end part including at least one third flexible distal portion, and a third intermediate part between said third proximal end part and said third distal end part, corresponding to and substantially aligned with, respectively, said first proximal end part, said first distal end part and said first intermediate part of said first cylindrical element.

13. The steerable instrument according to claim 8, wherein said third cylindrical element forms an inner cylindrical element, said first cylindrical element forms an intermediate cylindrical element, and said second cylindrical element forms an outer cylindrical element.

14. The steerable instrument according to claim 1, wherein said first cylindrical element, said second cylindrical element, and, where present, said third cylindrical element, are provided with one or more flexible portions throughout their lengths.

15. The steerable instrument according to claim 1, comprising a plurality of tangential spacers and a plurality of radial spacers as well as a plurality of longitudinal elements, wherein between each adjacent longitudinal elements at least one tangential spacer and at least one radial spacer are provided.

16. The steerable instrument according to claim 1, comprising a plurality of tangential spacers, a plurality of radial spacers and a plurality of longitudinal elements,
wherein said plurality of longitudinal elements comprises a first longitudinal element, a second longitudinal element, and a third longitudinal element,
wherein, in said first intermediate part,
a first open space is formed between said first longitudinal element and said second longitudinal element,
a second open space is formed between said second longitudinal element and said third longitudinal element,
wherein in the first open space at least one tangential spacer and at least one radial spacer are provided; and
wherein in the second open space an extended tangential spacer is provided, which extended tangential spacer extends substantially throughout the second open space.

17.-23. (canceled)

24. Method of manufacturing a steerable instrument for endoscopic and/or invasive type of applications, such as in surgery, the method comprising:
providing a first cylindrical element comprising a first proximal end part, a first distal end part including at least one first flexible distal portion, and a first intermediate part between said first proximal end part and said first distal end part;
forming at least one longitudinal element extending between said first proximal end part and said first distal end part and arranged for transferring a force from said first proximal end part to said at least one first flexible distal portion and thereby control bending of said at least one flexible distal portion;
forming at least one tangential spacer tangentially adjacent to said at least one longitudinal element, said at least one tangential spacer being tangentially attached to said at least one longitudinal element by at least one releasable attachment;
providing a second cylindrical element comprising a second proximal end part, a second distal end part including at least one second flexible distal portion, and a second intermediate part between said second proximal end part and said second distal end part;
forming at least one lip shaped portion defined by a first slot in a second wall of said second cylindrical element;
providing a third cylindrical element comprising a third proximal end part, a third distal end part including at least one third flexible distal portion, and a third intermediate part between said third proximal end part and said third distal end part;
sliding said cylindrical elements into one another, such that said cylindrical elements are substantially coaxially aligned;
applying a force onto said at least one lip shaped portion in a direction substantially perpendicular to said second wall of said second cylindrical element, thereby bending said lip shaped portion in a radial direction;
such that after bending said at least one lip shaped portion forms at least one radial spacer protruding into in an aperture in the tangential spacer;
and such that, after said releasable attachments have been released:
movement of said tangential spacer along both said longitudinal direction and in a tangential direction of the steerable instrument is confined by said at least one radial spacer.

25. Method according to claim 24, wherein said third cylindrical element is provided with at least one aperture or recess in a third wall of said third cylindrical element, and wherein after said bending said lip shaped portion engages with said aperture or recess.

26. Method according to claim 25, wherein said lip shaped portion is formed by:
forming said first slot such that said lip shaped portion is formed in said second wall and attached thereto via a beam having a beam axis, wherein said first slot extends around said lip shaped portion, except for where the lip shaped portion is attached to said beam, and further extending in a direction substantially parallel to said beam axis on either side of said lip shaped portion at one side of said beam; and
forming a second slot in said wall, said second slot extending in a direction substantially parallel to said beam axis at another side of said beam, said lip shaped portion further comprising a first part having a first width in a direction substantially parallel to said beam axis and a second part having a second width in a direction substantially parallel to said beam axis, wherein said first part is arranged between said beam and said second part, and wherein said second width is smaller than said first width,
wherein said second part engages with said aperture or recess of said third cylindrical element.

27. Method according to claim 24, further comprising releasing said at least one releasable attachment of said at least one tangential spacer.
